(19) United States
(12) Patent Application Publication
(10) Pub. No.: US 2008/0193468 A1
(43) Pub. Date: Aug. 14, 2008

(54) Method for Stimulating the Immune Response of Newborns

(75) Inventors: Ofer Levy, Jamaica Plain, MA (US); Michael Wessels, Brookline, MA (US); Richard Miller, Maplewood, MN (US); Mark Tomai, Woodbury, MN (US)

Correspondence Address: DAVID S. RESNICK, NIXON PEABODY LLP, 100 SUMMER STREET, BOSTON, MA, 02110-2131, US

(73) Assignees: CHILDREN'S MEDICAL CENTER CORPORATION, Boston, MA (US); 3M INNOVATIVE PROPERTIES COMPANY, Saint Paul, MN (US)

(21) Appl. No.: 11/661,746

(22) PCT Filed: Sep. 8, 2005
(86) PCT No.: PCT/US05/31904

§ 371 Date: Aug. 21, 2007

Related U.S. Application Data
(60) Provisional application No. 60/607,833, filed on Sep. 8, 2004.
(60) Provisional application No. 60/692,325, filed on Jun. 20, 2005.
(60) Provisional application No. 60/694,267, filed on Jun. 27, 2005.

(51) Int. Cl. A61K 39/00; A61K 31/437; A61K 31/7105; A61P 31/00

(52) U.S. Cl. 424/184.1; 514/293; 514/44

(57) ABSTRACT

The present invention is based on the surprising discovery that agonists of TLR8 are uniquely efficacious in enhancing (e.g. inducing) the immune response of newborns. Thus, agonists of TLR8 serve as both vaccine adjuvants and as adjunctive therapies for acute infection in newborns, preferably the agonist is a TLR8-selective agonist. The immune response induced, or enhanced, in the neonatal host can be, for example, a cytokine immune response and/or a humoral immune response (e.g., antigen-specific).

### CROSS-REFERENCE TO RELATED APPLICATIONS

This application claims the benefit under 35 U.S.C. §119(e) of the U.S. Provisional Application Ser. No. 60/607,833, filed on Sep. 8, 2004; U.S. Provisional Application Ser. No. 60/692,325, filed Jun. 20, 2005 and U.S. Provisional Application Ser. No. 60/694,267, filed on Jun. 27, 2005, the entire contents of which are incorporated herein by reference.

### GOVERNMENT SUPPORT

This invention was supported by the National Institutes of Health-NIH Grant Nos. K08 AI50583-01 and N01 AI 25495. The government of the United States has certain rights thereto.

### BACKGROUND OF THE INVENTION

Newborns suffer a higher frequency and severity of microbial infection than older children and healthy middle-aged adults (Klein, J., and J. Remington. 2001. Current Concepts of Infections of the Fetus and Newborn Infant. In Infectious Diseases of the Fetus and Newborn Infant. J. Remington, and J. Klein, eds. W.B. Saunders Company, Philadelphia, p. 1.). Invasive neonatal infections are associated with high morbidity and mortality, necessitating a conservative diagnostic and therapeutic approach toward newborns presenting with fever or other signs of infection. However, newborns have a relatively poor response to most vaccines posing substantial challenges to preventing infections in this susceptible population. The poor neonatal response to most vaccines has been attributed to immaturity of the acquired immune system at birth (Zinkernagel, R. M. 2001. Maternal antibodies, childhood infections, and autoimmune diseases.[see comment]. New England Journal of Medicine 345:1331).

Over the past decade, there has been rapid progress in defining the molecular mechanisms by which the human host's innate immune system recognizes and responds to a variety of microbe-associated molecules (Hoffman et al. 1999. Science 284:1313). These microbial products activate host cells via Toll-like receptors (TLRs) (Landmann et al. 2000. Microbes & Infection. 2:295). In addition to microbial products, the synthetic imidazoquinolines (Stanley. 2002. Clinical & Experimental Dermatology. 27:571), imiquimod and its congener resiquimod (R-848), activate murine cells via TLR7 (Hemmi et al. 2002. Nature Immunology. 3:196); whereas in human cells, resiquimod also activates via TLR8 (Jurk et al. 2002. Nature Immunology. 3:499). Both imiquimod, which has been approved as a topical immunomodulatory therapy for human papilloma virus infection, and resiquimod enhance release of Th1-type cytokines including TNF-α (Harandi et al. 2003. Current Opinion in Investigational Drugs. 4:156; Jones. 2003. Current Opinion in Investigational Drugs. 4:214).

Innate immune recognition of microbial products at normally sterile sites such as blood begins with fluid-phase recognition of microbial products by host factors that can greatly enhance or inhibit ligand-induced cellular signaling. For example, by efficiently delivering LPS monomers to the endotoxin receptor complex composed of membrane CD14, TLR4, and MD2, the LPS-binding protein (LBP) greatly enhances LPS-induced inflammatory responses, accounting for the ability of human plasma/serum to greatly amplify LPS-induced inflammatory activity (Ulevitch and Tobias. 1999. Current Opinion In Immunology 11:19). At higher concentrations, however, LBP serves to shuttle LPS to plasma lipoproteins and thereby detoxify it (Vreugdenhil et al. 2003. Journal of Immunology. 170:1399). Soluble CD14 (sCD14) is also a constituent of human plasma that modulates the activity of LPS upon host cells (Kitchens et al. 2001. Journal of Clinical Investigation. 108;485). Less is known about plasma factors that may modulate signaling by other TLR ligands.

Engagement of TLRs activates cytosolic signaling via a family of adapter molecules including MyD88 and TIRAP (Akira. 2003; 278:38105). Following TLR activation, these adapter molecules recruit the IL-1R-associated kinase IRAK-4 activation of which initiates a cascade leading to phosphorylation of MAP kinases, translocation of nuclear factor-κB, and consequent transcription of multiple genes, including that encoding TNF-α (Akira. 2003. Curr Op Immunol 15:5).

Despite substantial progress in understanding TLR-activated signaling at the molecular level, very little is known about the expression and function of these pathways at birth. The newborn immune system has been generally considered “functionally immature”, and some studies of neonatal and adult leukocytes with respect to release of cytokines upon stimulation in vitro have suggested that newborn responses are impaired (Cohen et al. 1995. Journal Of Immunology 155:5337; Bessler et al. 2001. Biology of the Neonate. 80:186). A recent study has described a correlation between reduced responsiveness of newborn mononuclear cells to LPS and reduced MyD88 expression (Yan et al. 2004. Infection & Immunity 72:1223).

There is a need in the art to better understand the mechanistic differences between the ability of newborns versus the ability of infants, children and adults to mount an immune response against foreign pathogens. In this manner a means for stimulating the immune response of neonates can be developed. In addition, there is a need in the art for methods of vaccination that are successful in newborns. The ability to vaccinate a child at birth would not only significantly reduce morbidity and mortality of neonates due to infection, it would also avoid infections in infants, children, and adults.

### SUMMARY OF THE INVENTION

The present invention is based on the surprising discovery that agonists of TLR8 are uniquely efficacious in enhancing (e.g. inducing) the immune response of newborns. Thus, agonists of TLR8 serve as both vaccine adjuvants and as adjunctive therapies for acute infection in newborns. The immune response induced, or enhanced, in the neonatal host can be, for example, a cytokine immune response and/or a humoral immune response (e.g., antigen-specific).

The invention provides for a method for enhancing the immune response of a newborn comprising administering to said newborn an effective amount of a compound or agent that is an agonist of Toll-Like receptor 8 (TLR8). In some cases, the TLR8 agonist may be an agonist of TLR7 and Toll-Like receptor 8 (TLR7/8). Preferably, the compound or agent is a TLR8-selective agonist. The immune response to be enhanced, for example, can be a Th1 immune response, an innate immune response, a local immune response, a mucosal immune response, or a systemic immune response.

Any agonist of TLR8 can be used in methods of the invention. In one embodiment, the TLR8 agonist is an imidazoquinoline compound. In one preferred embodiment, the compound is resiquimod. In another embodiment, the TLR8 agonist may be a tetrahydroimidazoquinoline amine In a preferred embodiment, the compound is 4-amino-2-(ethoxymethyl)-α,α-dimethyl-6,7,8,9-tetrahydro-1H-imidazo[4,5-c]quinoline-1-ethanol. In other preferred embodiments, the TLR8 agonist may be a thiazoloquinoline amine. Additionally, any combination of TLR8 agonist may be used.

In another embodiment, the TLR8 agonist is single stranded ribonucleic acid (ssRNA).

In one embodiment, the TLR8 agonist is a compound or agent that binds to TLR8 thereby inducing cell signaling mediated by TLR8. Alternatively, the TLR8 agonist is a compound or agent that induces the activity of a downstream signaling molecule that is activated by TLR8.

The invention further provides for a method of preventing or treating an acute infection in a newborn comprising administering to said newborn an effective amount of a compound or agent that is an agonist of TLR8, wherein said agonist enhances the immune response of the newborn.

In one embodiment, the acute infection to be prevented or treated by methods of the invention is a bacterial infection.

In one embodiment, the acute infection to be prevented or treated by methods of the invention is a viral infection.

In one embodiment, the acute infection to be prevented or treated by methods of the invention is a fungal infection.

In one embodiment, the acute infection to be prevented or treated by methods of the invention is a parasitic infection.

In one preferred embodiment, the TLR8 agonist administered for treatment or prevention of the acute infection is co-administered with an additional therapeutic agent. The agonist can be administered concurrently, before, or after, administration of the additional therapeutic agent.

The invention further provides for a method for vaccinating a newborn against an infection or disorder comprising administering to said newborn an effective amount of a compound or agent that is an agonist of TLR8 and administering to said newborn a vaccine, wherein said agonist enhances the newborn's immune response to an antigen in said vaccine.

The TLR8 agonist can be used as an adjuvant to enhance the immune response to any vaccine antigen, e.g. bacterial, viral or even cancer.

In one embodiment, the TLR8 agonist used in methods of the invention is an imidazoquinoline compound. In one preferred embodiment, the compound is resiquimod. In another embodiment, the TLR8 agonist may be a tetrahydroimidazoquinoline amine In a preferred embodiment, the compound is 4-amino-2-(ethoxymethyl)-α,α-dimethyl-6,7,8,9-tetrahydro-1H-imidazo[4,5-c]quinoline-1-ethanol. In other preferred embodiments, the TLR8 agonist may be a thiazoloquinoline amine. Additionally, any combination of TLR8 agonist may be used.

In another embodiment, the TLR8 agonist is ssRNA.

In one embodiment, the TLR8 agonist is a compound or agent that binds to TLR8 thereby inducing cell signaling mediated by TLR8. Alternatively, the TLR8 agonist is a compound or agent that induces the activity of a downstream signaling molecule that is activated by TLR8.

In one embodiment, the agonist is administered concurrently with said vaccine or therapeutic agent.

In another embodiment, the agonist is administered before said vaccine or therapeutic agent.

In still another embodiment, the agonist is administered after said vaccine or therapeutic agent.

### BRIEF DESCRIPTION OF FIGURES

FIGS. 1A to 1E show impaired ligand-induced TNF-α release in newborn cord blood in response to bacterial lipopeptides (BLPs), lipopolysaccharide (LPS), and imiquimod but preserved response to resiquimod. TNF-α release from newborn cord blood and adult peripheral blood was measured after a 5-hour incubation with FIG. 1A, triacylated BLP (TLR1/2), FIG. 1B, MALP (TLR2/6), FIG. 1C, LPS (TLR4), FIG. 1D, imiquimod (TLR7), and FIG. 1E, resiquimod (TLR7/8). Ligand structures are indicated above each panel with the N-acyl-S-diacylglycerylcysteine of BLP depicted as a rectangle, and the Kdo and GlcN sugars of Re595 LPS indicated as open and filled hexagons, respectively. The number of independent determinations (N) is indicated in the symbol legend. *p<0.05, **<0.01, ***<0.001, ****<0.0001.

FIGS. 2A to 2C show that cord blood derived from both Caesarian-section and vaginal deliveries demonstrates impaired tBLP- and LPS- but preserved resiquimod-induced TNF-α release. Blood was incubated with the indicated concentrations of tBLP (FIG. 2A), LPS (FIG. 2B), or resiquimod (FIG. 2C), for 5 hours then assayed for TNF-α by ELISA. Adult controls are shown for comparison. N=3-5 study subjects in each category.

FIGS. 3A to 3C show lower magnitude, but similar kinetics, of tBLP-(FIG. 3A) and LPS-(FIG. 3B), induced TNF-α release in newborn cord vs. adult peripheral blood. In contrast, newborns mount an equivalent TNF-α response to resiquimod (FIG. 3C). Results are representative of one of three similar experiments.

FIGS. 4A to 4C show ligand-induced monocyte TNF-α synthesis. FIG. 4A, Relative ligand-induced intracellular TNF-α production by monocytes in blood as measured by flow cytometry. Whole blood was incubated with tBLP (10 μg/mL), LPS (10 ng/mL), or resiquimod (1 μg/mL) for 4 hours then monocytes were stained with a phycoerythrin-conjugated anti-TNF-α. Intracellular TNF-α production was calculated as described in Example 1(N=3-4). FIG. 4B, LPS- and resiquimod-induced TNF-α release from isolated newborn and adult monocytes tested in autologous serum (N=3), FIG. 4C, Monocyte TNF-α mRNA synthesis in response to LPS and resiquimod. Whole blood was incubated with buffer, LPS (100 ng/mL), or resiquimod (10 μg/mL) for 6 hours, monocyte total RNA was subjected to TNF-α real time PCR (RT-PCR) as described in Example 1 (N=3). *p<0.05.

FIGS. 5A to 5B show phosphorylation of monocyte p38 MAP kinase upon stimulation of newborn or adult blood with TLR ligands. Newborn or adult whole blood was stimulated with LPS (10 ng/mL) (FIG. 5A) or resiquimod (1 μg/mL) (FIG. 5B) for the indicated times. Intracellular phospho-p38 was detected by flow cytometry with a phycoerythrin-conjugated mAb. Data represent the difference of p-p38 mean fluorescent intensity (MFI) between stimulated and unstimulated monocytes at each time point. Results representative of three similar experiments (N=3) are shown.

FIGS. 6A to 6C show similar basal expression of TLRs and TLR-related molecules in newborn and adult monocytes. FIG. 6A, Basal monocyte mRNA expression by RT-PCR Analysis (N=7-11); FIG. 6B, Basal total TLR2 protein expression by ELISA (N=3); FIG. 6C, Basal monocyte surface expression of TLR2, TLR4 and CD14 by flow cytometry of whole blood (N=8-15).

FIGS. 7A to 7B show modulation of TLR and CD14 surface expression upon stimulation of newborn and adult monocytes. FIG. 7A, Percent change in surface expression of monocyte CD14, TLR1 and TLR2 5 min after the addition of tBLP (10 μg/mL) to whole blood (N=6);*p<0.05. FIG. 7B. Monocyte surface expression of CD14 after stimulation of whole blood with LPS (100 ng/mL) for the indicated times, (N=3-5) p<0.05 by ANOVA.

FIG. 8 shows the differences in the ability of newborn and adult plasma to modulate ligand-induced TNF-α release. Newborn or adult hemocytes were washed and resuspended in autologous or heterologous plasma prior to addition of TLR ligands and measurement of TNF-α release. For the purposes of comparison, the effects of heterologous plasma on ligand-induced TNF-α release were expressed as a “Modulation Index” as shown in the example provided in the inset (“Method of Data Analysis”). In this example, the presence of adult plasma in the heterologous condition (N cells/A plasma) resulted in amplification of the ligand-TNF-α dose-response curve such that 0.1 μg/mL of ligand yielded as much TNF-α release as 10 μg/mL did under the autologous condition (N cells/N plasma), indicating a modulation index of 100 (i.e., 100-fold increased activity in the presence of adult plasma). Such analysis was performed for each of the TLR ligands tested: tBLP, MALP, LPS, imiquimod, and resiquimod. For all TLR ligands except resiquimod, adult plasma increased TNF-α release from newborn hemocytes whereas newborn plasma reduced TNF-α release from adult hemocytes (N=3-4, p<0.05 by Mann-Whitney test for all comparisons except resiquimod).

FIGS. 9A to 9B show that differences in sCD14 concentrations between newborn and adult plasma do not account for discrepancies in tBLP- or LPS-induced TNF-α release. (FIG. 9A) The concentration of sCD14 is lower in newborn than adult plasma (439±59 vs. 1109 ±30 ng/ml). FIG. 9B, however addition of either 500 or 1,000 ng of purified sCD14 per mL of newborn blood (i.e., final [sCD14] approximating or exceeding that in adults) did not restore tBLP or LPS-induced TNF-alpha release. *p<0.05, **p<0.01, ***p<0.001 by Student's t test for adult compared to newborn.

FIGS. 10A to 10D confirm that among the TLR ligands, those that activate via TLR8 are uniquely effective at fully activating neonatal cells. TNF-α release from newborn cord blood and adult peripheral blood was measured after a 5-hour incubation with FIG. 10A, Loxoribine (TLR7), FIG. 10B, imiquimod (TLR7), FIG. 10C, resiquimod (TLR7/8) and FIG. 10D, ssRNA (TLR8). Single stranded ribonucleic acid (ssRNA) tested in this study was ssRNA40/LyoVec purchased from InvivoGen (San Diego, Calif.) comprised of single-stranded GU-rich oligonucleotide (5′-GsCsCsCsGsUsCsUsGsUsUsGsUsGsUsGsAsCsUsC-3′; where “s” depicts a phosphothioate linkage) complexed with the cationic lipid “LyoVec” (to protect the RNA from degradation and enhance is uptake by immune cells). The guanosine analog loxoribine (TLR7 ligand) was purchased from InvivoGen.

FIG. 11 shows the structures of two imdazoquinoline TLR agonists: imiquimod (TLR7) and resiquimod (TLR 7/8). Imiquimod is an agonist at TLR7 receptors whereas resiquimod is an agonist at both TLR7 and TLR 8. Resiquimod is ˜100-fold more potent than imiquimod.

FIG. 12A to 12C show agonists of TLR8 (±TLR7) effectively induce TNF-α and IL-12 release from human neonatal blood, whereas agonists of TLR 2/6, -4, -7 or -9 only do not. FIG. 12A shows freshly collected neonatal cord (open bars) or adult peripheral (black bars) blood (citrate) was incubated with TLR agonists for 5 h. After stopping the reaction with ice-cold culture medium, the extracellular fluid was collected for measurement of TNF-α by ELISA (R & D Systems). FIG. 12B shows TNF-α release induced by TLR agonists in heparinized blood with overnight incubation. Freshly collected neonatal cord (open bars) or adult peripheral (black bars) blood (heparin) was incubated with TLR agonists overnight. After stopping the reaction with ice-cold culture medium, the extracellular fluid was collected for measurement of TNF-α by ELISA (R & D Systems). FIG. 12C shows IL-12 release induced by TLR agonists after 19 h incubation.

FIG. 13 shows agonists that activate via TLR8 (±TLR7) induce equivalent TNF-α secretion from neonatal and adult PBMCs cultured in autologous serum, but agonists that activate via TLR7 only do not. TLR agonists were added to PBMCs cultured in autologous serum for 5 hours after which the extracellular medium was collected for measurement of TNF-α by ELISA

FIG. 14A to 14C show the TLR 7/8 agonist resiquimod induces substantial IL-12 release from neonatal and adult monocytes, but LPS (TLR4) does not. Neonatal or adult PBMCs were adhered to plastic wells and cultured in fresh autologous serum. Cells were exposed to TLR agonists for 4 h (FIG. 14A) or 24 h (FIG. 14B and FIG. 14C) after which the extracellular medium was recovered for IL-12 p70 ELISA.

FIG. 15A to 15D show the TLR7/8 agonist resiquimod induces upregulation of CD40 expression in neonatal myeloid dendritic cells (mDCs) whereas the TLR7 agonist imiquimod does not. FIG. 15A shows Newborn cord blood was incubated with imiquimod (250 μM) and FIG. 15B with resiquimod (50 μM) for 19 hours. After lysis of red blood cells and fixation, mDCs were identified as 1in1-/HLA-DR+/CD11c+ cells using four color flow cytometry (BD BioSciences) and CD40 was measured using a PE-conjugated mAb. FIG. 15C shows percent increase in CD40 expression index of mDC in whole blood after 19 h incubation. FIG. 15D shows the ratio of newborn to adult TLR lligand-induced CD40 expression index of mDCs.

FIG. 16A to 16C show TLR8 (±TLR 7) agonists effectively induce CD40 expression on neonatal myeloid dendritic cells, whereas TLR 1/2, TLR 2/6, and TLR4 and TLR7 agonists do not. Newborn cord or adult peripheral blood was incubated with TLR agonists at 37° C. for 19 hours. mDCs were identified by flow cytometry and the level of expression of CD40 was measured using a PE-conjugated mAb. FIG. 16A shows the percent of mDCs positive for CD40. FIG. 16B shows data expressed as an “expression index” representing the product of the mean fluorescent intensity per cell and the % mDCs positive for CD40. FIG. 16C shows TLR ligand-specific CD40 mean fluorescent intensity (MFI) of mDCs.

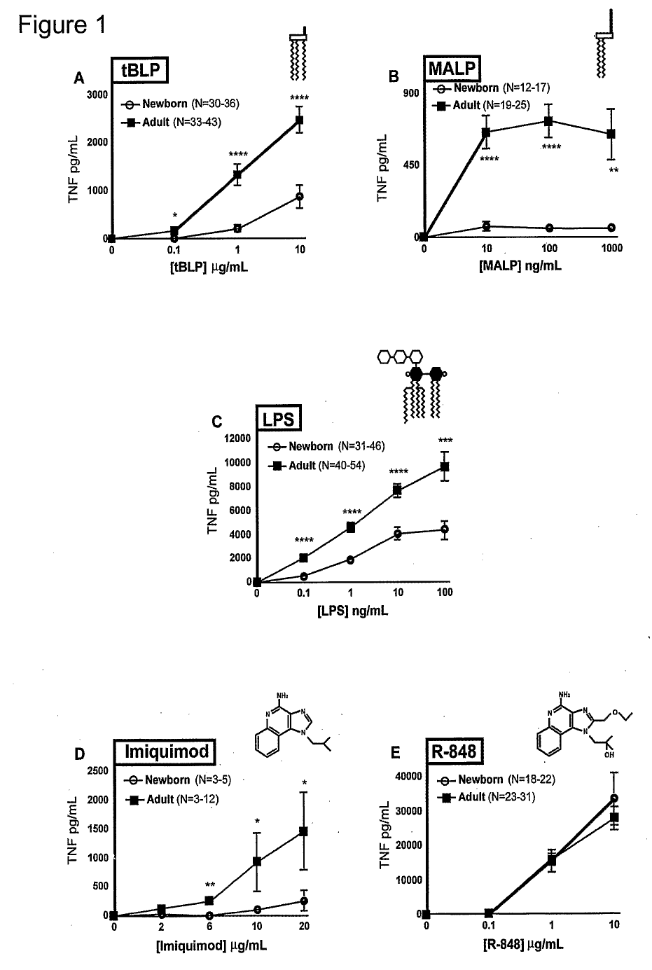

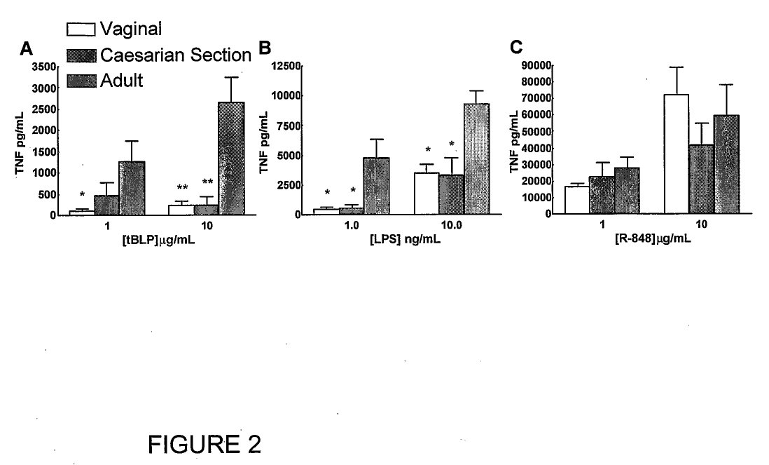

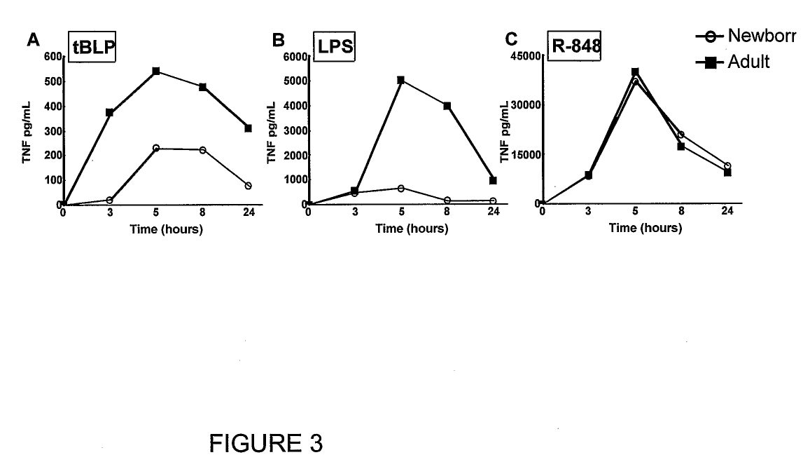

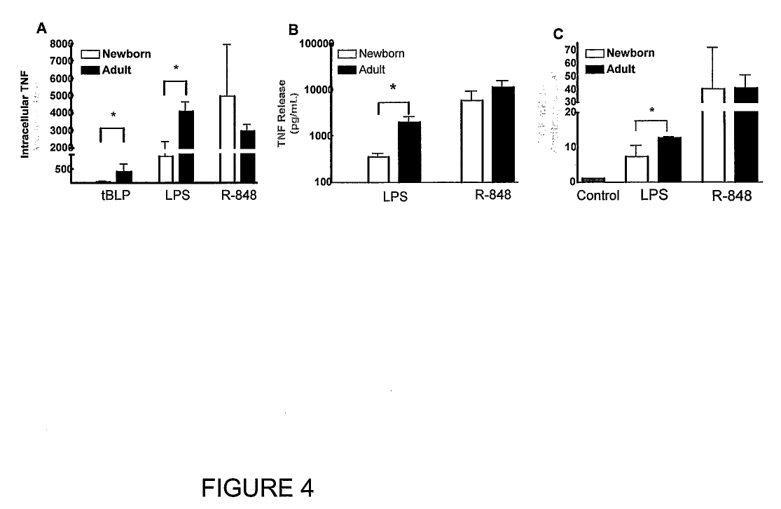

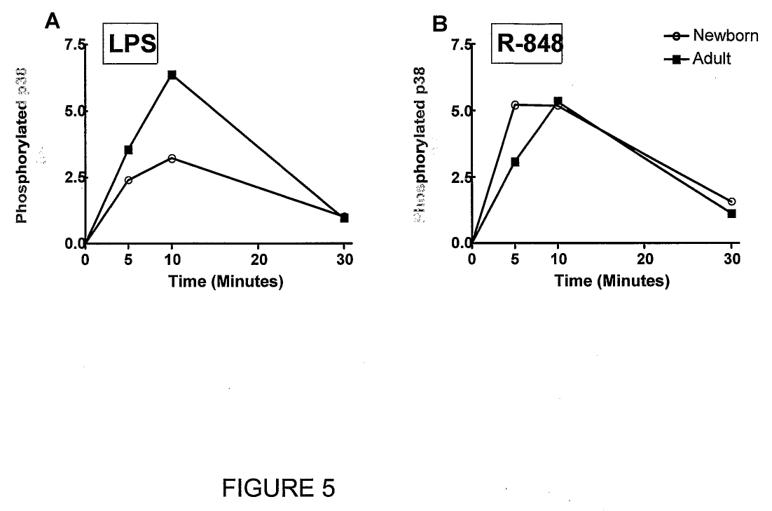

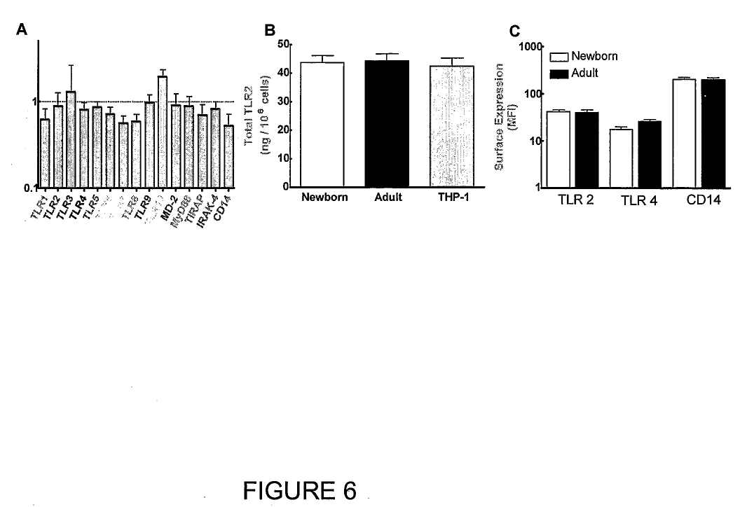

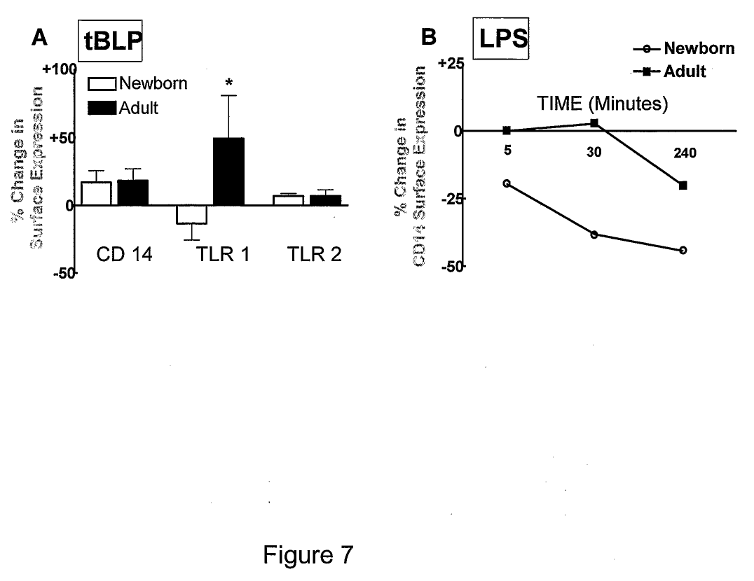

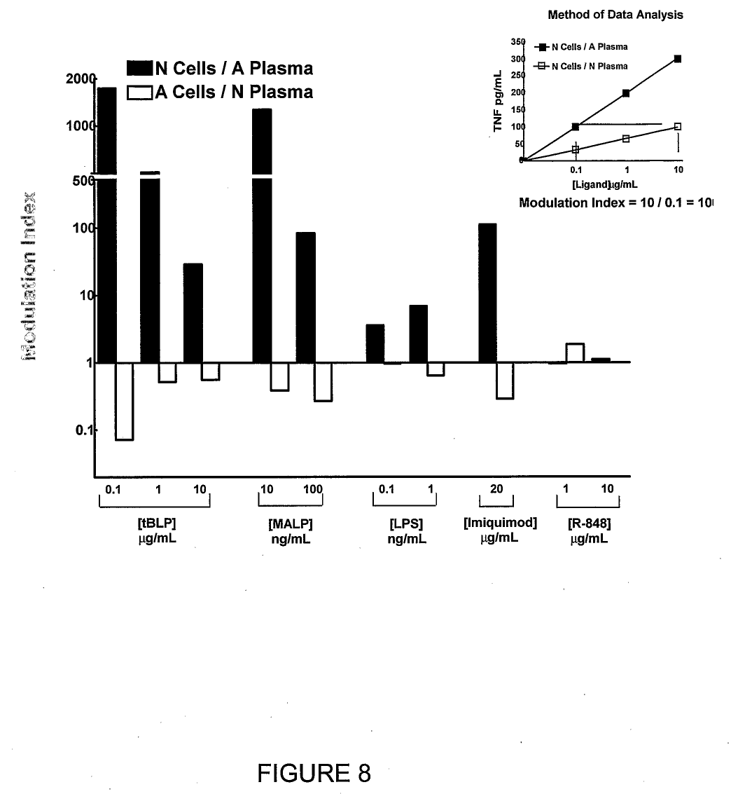

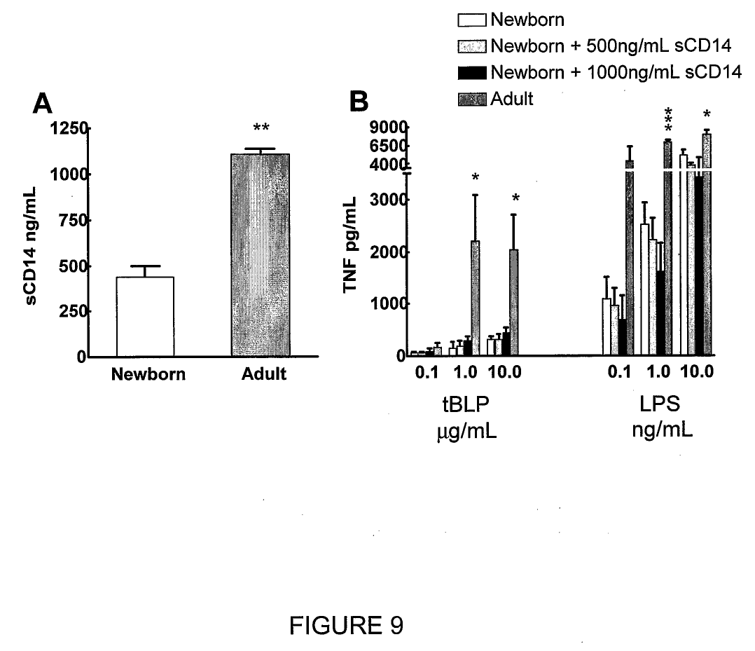

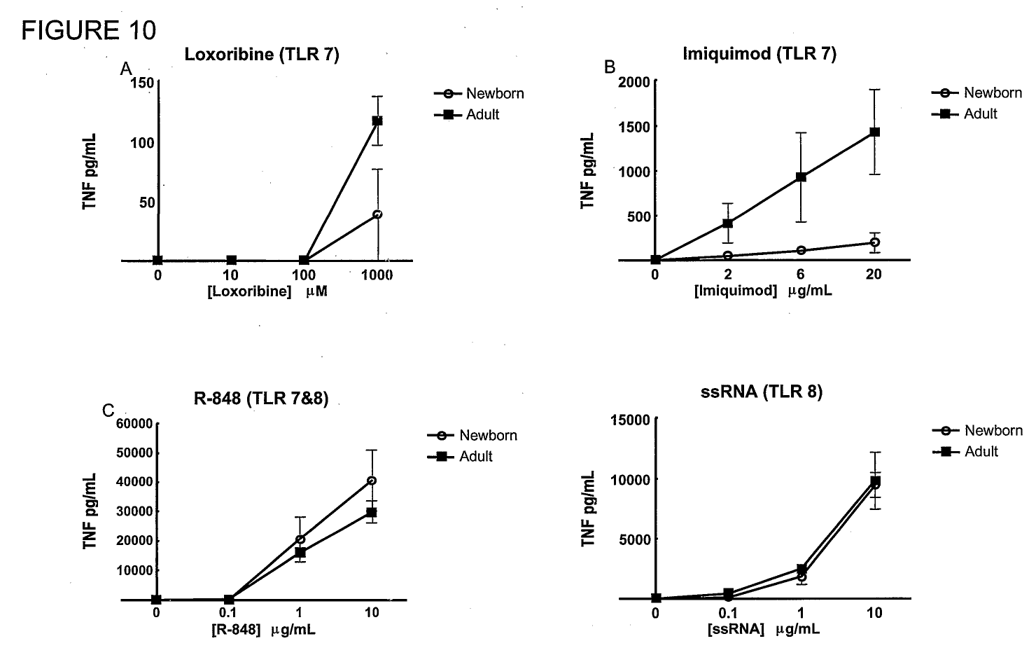

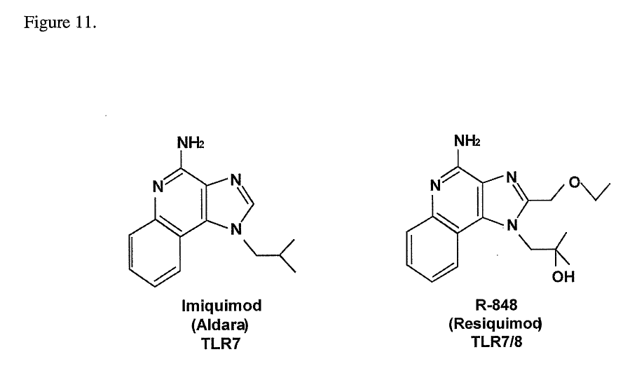

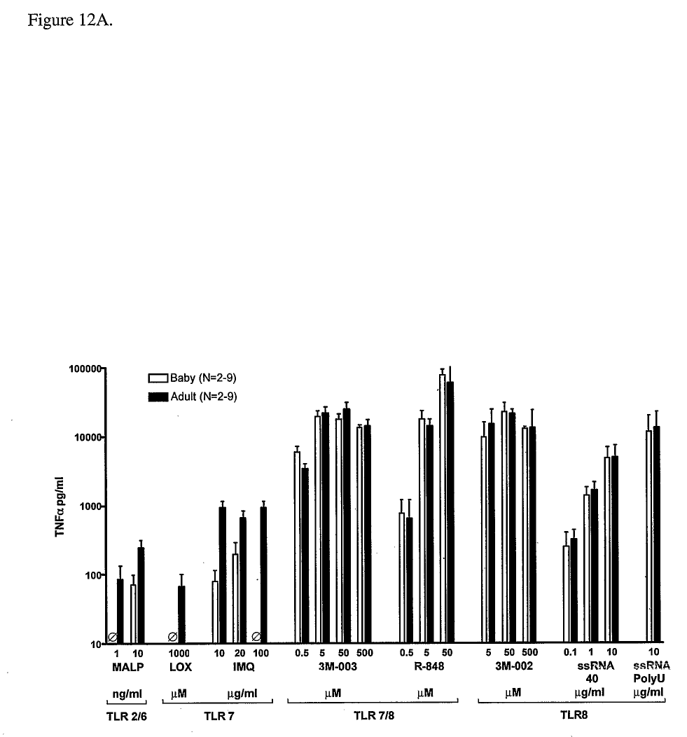

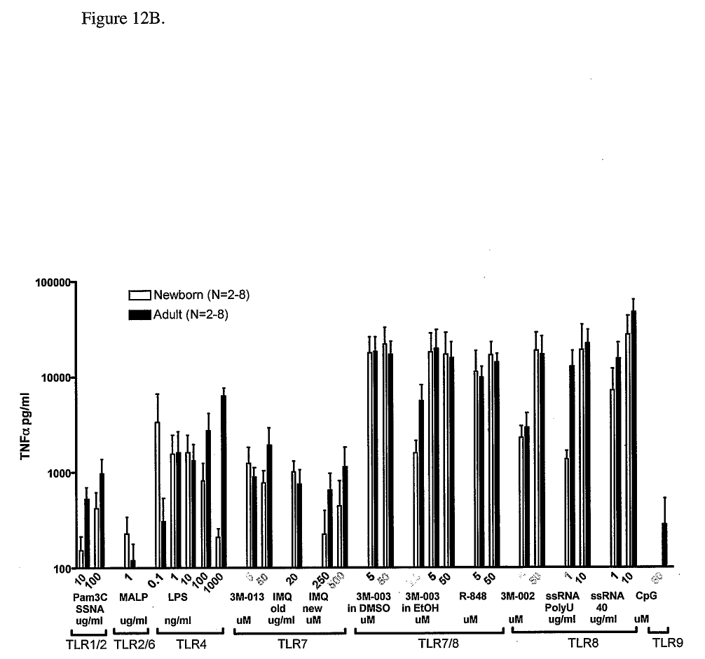

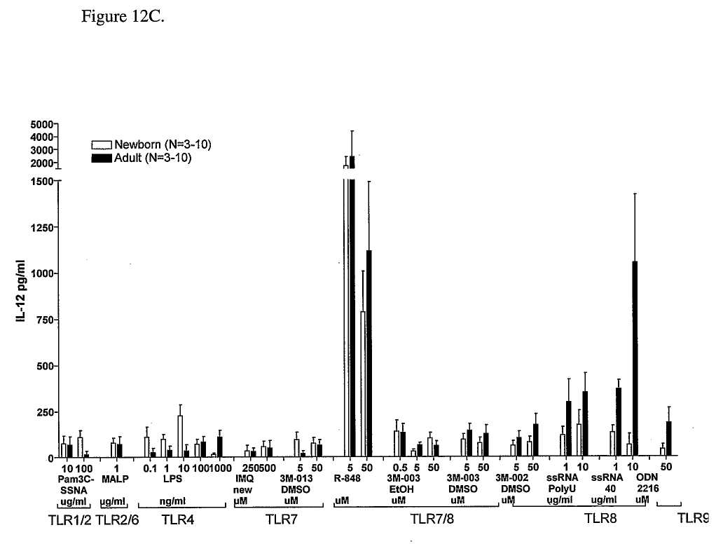

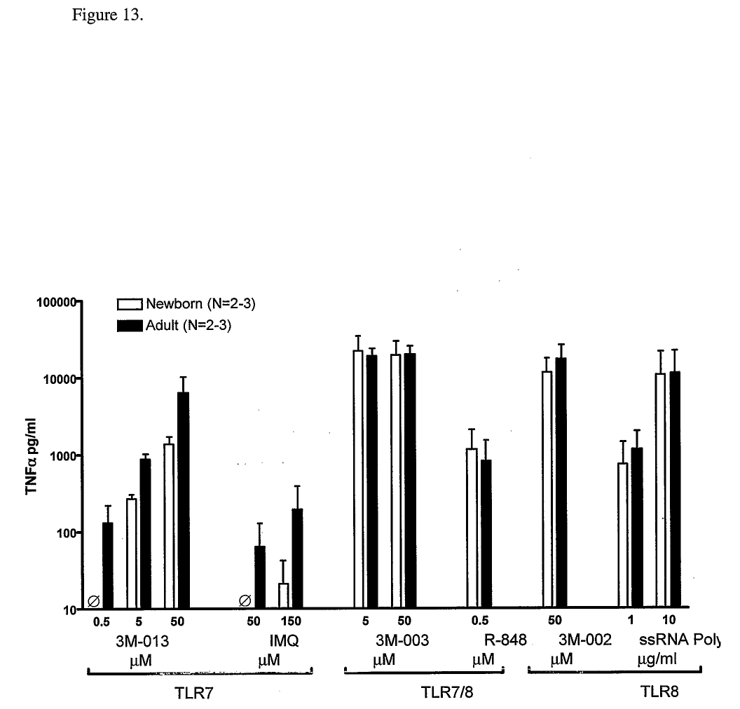

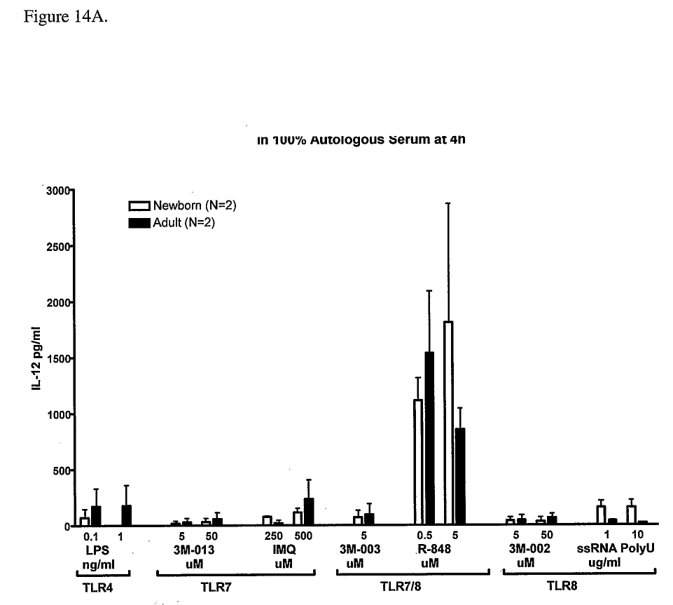

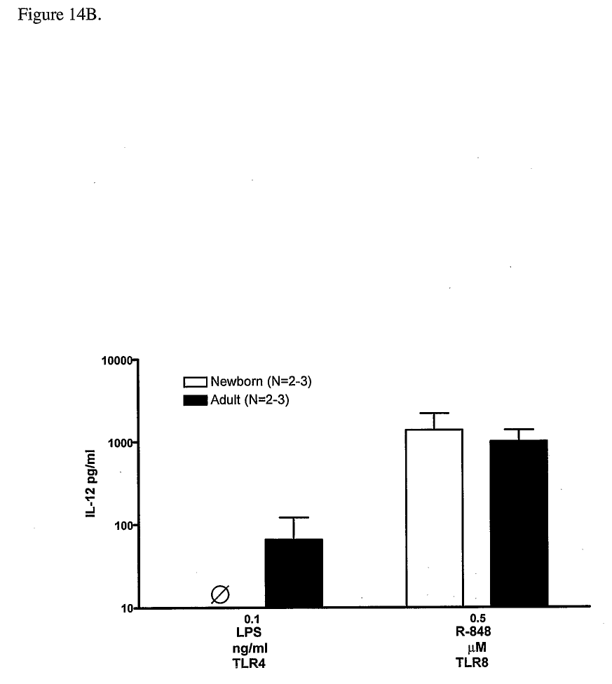

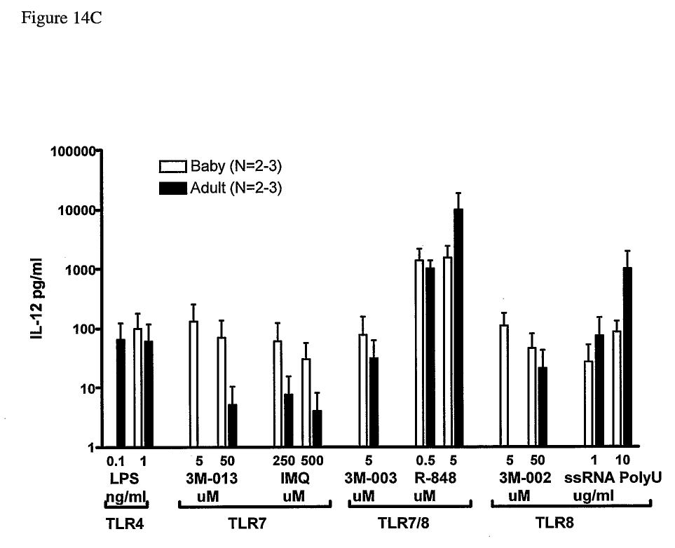

### DETAILED DESCRIPTION OF THE INVENTION

The present invention provides methods for inducing or enhancing the immune response of newborns. In some cases, the methods comprise administration of a compound or agent that is an agonist of both Toll-like Receptors 7 and 8 (TLR7/8). In other cases, the methods include administering a compound or agent that is a TLR8-selective agonist.

### Definitions

The following definitions are provided for specific terms which are used in the following written description.

As used herein, “Toll-like receptor 8” or “TLR8” or “Toll-like receptors 7 and 8” or “TLR7/8” refers to a receptor that is a member of the Toll-like receptor (TLR) family. TLRs are transmembrane proteins characterized by an extracellular leucine-rich domain and a cytoplasmic tail that contains a conserved region called the Toll/IL-1 receptor (TIR) domain. TLRs are predominantly expressed in tissues involved in immune function, such as spleen and peripheral blood leukocytes, as well as those exposed to the external environment such as lung and the gastrointestinal tract. The natural ligand of TLR8 is currently unknown, however, TLR8 is known to bind some small molecules such as resiquimod, an imidazoquinoline compound with antiviral activity. Non-limiting examples of TLR8 receptors are found in Genebank at accession numbers AAF64061, AAF78036, AAK62677, AAQ88663, NP_{—}057694 and NP_{—}61952. The term “TLR8” is also intended to encompass homologues and allelic variants thereof.

As used herein, the term “agonist” refers to any compound or agent that stimulates or increases activity mediated by a receptor (e.g., a TLR). Thus, the term “TLR8 agonist” includes any compound or agent that stimulates or increases TLR8 activity. A TLR8 agonist can be an agent that binds to TLR8 thereby inducing signal transduction mediated by the receptor. The term TLR8 agonist, as used herein, also encompasses compounds or agents that induce the activity of a downstream signaling molecules that are activated by TLR8. TLR8 agonists include, for example, antibodies, as defined herein, and molecules having antibody-like function such as synthetic analogues of antibodies, e.g., single-chain antigen binding molecules, small binding peptides, or mixtures thereof. Agents having agonist activity also includes small organic molecules, natural products, peptides, aptamers, peptidomimetics, DNA and RNA.

As used herein, the term “TLR8-selective agonist” refers to a TLR8 agonist that stimulates TLR8 to a significantly greater degree than it stimulates any other TLR. Thus, while “TLR8-selective agonist” may refer to a compound or agent that acts as an agonist of TLR8 and for no other TLR, it may also refer to a compound or agent that acts primarily as an agonist of TLR8, but also induces minor levels of activity mediated by another TLR.

As used herein, the singular (e.g., “a,” “an,” “the,”) includes the plural. Thus, for example, the singular term “TLR7/8 agonist” also includes the plural “TLR7/8 agonists.”

As used herein, the terms “TLR8 activity” refers to TLR8-mediated signal transduction.

As used herein, the term “antibody”, includes human and animal mAbs, and preparations of polyclonal antibodies, as well as antibody fragments, synthetic antibodies, including recombinant antibodies (antisera), chimeric antibodies, including humanized antibodies, anti-idiotopic antibodies and derivatives thereof.

As used herein, the term “administering” to a patient (i.e. newborn) includes dispensing, delivering or applying an active compound or agent in a pharmaceutical formulation to a subject by any suitable route for delivery of the active compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route. The agents may, for example, be administered to a comatose, anesthetized or paralyzed subject via an intravenous injection. Specific routes of administration may include topical application (such as by eyedrops, creams or erodible formulations to be placed under the eyelid, intraocular injection into the aqueous or the vitreous humor, injection into the external layers of the eye, creams or erodable formulations that can be applied to dermal and mucosal tissues, such as via subconjunctival injection, parenteral administration or via oral routes. The term “administering” to a patient (i.e. newborn) is also intended to include administration to a pregnant mother, such that the compound or agent crosses the placenta and is delivered to the neonatal host indirectly.

As used herein, “effective amount” of a compound or agent is an amount sufficient to achieve a desired therapeutic or pharmacological effect, such as an amount sufficient to induce the activity of TLR8. An effective amount of a compound or agent as defined herein may vary according to factors such as the disease state and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

A therapeutically effective amount or dosage of an agent may range from about 100 ng/kg to about 50 mg/kg body weight, although in some embodiments the agent may be administered in a dose outside this range. For example, an agent may be administered in a dose ranging from about 0.001 to 30 mg/kg body weight, with other ranges of the invention including about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, and 5 to 6 mg/kg body weight. The skilled artisan will appreciate that certain factors may influence the dosage required to effectively treat a subject, including but not limited to the severity of the disease or disorder, the general health of the subject, and other diseases present. Moreover, treatment of a subject with a therapeutically effective amount of an active compound can include a single treatment or a series of treatments. In one example, a subject is treated with an agent in the range of between about 0.1 to 20 m/kg body weight, one time per week for between about 1 to 10 weeks, alternatively between 2 to 8 weeks, between about 3 to 7 weeks, or for about 4, 5, or 6 weeks. It will also be appreciated that the effective dosage of an agent used for treatment may increase or decrease over the course of a particular treatment. An agonist can be administered before, concurrently with, or after administration of another agent (e.g., an antigen).

Unless otherwise indicated, reference to a compound or agent can include the compound or agent in any pharmaceutically acceptable form, including any isomer (e.g., diastereomer or enantiomer), salt, solvate, polymorph, and the like. In particular, if a compound is optically active, reference to the compound or agent can include each of the compound's or agent's enantiomers as well as racemic mixtures of the enantiomers.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “patient” or “subject” or “animal” or “host” refers to any “newborn” mammal. The patient is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the terms “newborn” or “neonate” refer to a baby that is 0-28 days old.

As used herein, the terms “enhance” and/or “enhancing” refer to the strengthening (augmenting) of an existing immune response to a pathogen in a neonatal host. The term also refers to the initiation of (initiating, inducing) an immune response to a pathogen in a newborn. Some pathogens include, for example, bacteria (e.g., Group B streptococcus, Bordetella pertussis, Bordetella parapertussis, bronchiseptica, Listeria monocytogenes, Bacillus anthracis, S. pneumoniae, N. meningiditis), viruses (e.g., hepatitis, measles, poliovirus, human immunodeficiency virus, influenza virus, parainfluenza virus, respiratory syncytial virus, herpes simplex virus), mycobacteria (e.g. M. tuberculosis and non-tuberculous myobacteria), parasites (Leishmania, Schistosomes, Trypanosomes, toxoplasma, pneumocystis) and fungi (e.g., Candida spp., Cryptococcus, Coccidiodes, Aspergillus spp.), as well as others.

Various aspects of the invention are described in further detail in the following subsections:

### TLR8 Agonists

The therapy described herein comprises administering to a newborn an agonist of TLR8, preferably a TLR8-selective agonist, such that the immune response of a newborn is stimulated. The TLR8 agonist can be administered before, concurrently with, or after administration of another agent. For example, the agonist can be administered with a vaccine to enhance the immune response of the newborn to the vaccine antigen. Alternatively the agonist can be administered before, concurrently with, or after administration of an additional therapeutic agent. When another agent is administered and the agents are administered at different times, they are preferably administered within a suitable time period to provide substantial overlap of the pharmacological activity of the agents. The skilled artisan will be able to determine the appropriate timing for co-administration of the agonist and the additional agent depending on the particular agents selected and other factors.

The TLR8 agonist can be DNA, RNA, a small organic molecule, a natural product, protein (e.g., antibody), peptide or peptidomimetic. Agonists can be identified, for example, by screening libraries or collections of molecules, such as, the Chemical Repository of the National Cancer Institute, as described herein or using other suitable methods. Suitable screening methods that can be used to identify TLR8 agonists for use in the present invention, as well as known TLR8 agonists, are described in U.S. Patent Application No.'s 20040132079 and 20030139364 and PCT publication WO 03/094836, which are herein incorporated by reference in their entirety.

In one preferred embodiment, the agonist is a small molecule immune response modifier (IRM) compound. Generally, IRMs include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}2 cytokines, such as IL-4 and IL-5.

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,627,638; 6,331,539; 6,376,669; 6,440,992; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. 2004/0091491; 2004/0147543; 2004/0176367; and 2005/0021334; International Publication Nos. WO 2005/18551, WO 2005/18556, and WO 2005/20999; and U.S. Provisional Patent Ser. No. 60/651585, the entire contents of which are incorporated herein by reference.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

In one preferred embodiment, the agonist is ssRNA, such as ssRNA40/LyoVec, which is comprised of single-stranded GU-rich oligonucleotide (5′-GsCsCsCsGsUsCsUsGsUsUsGsUsGsUsGsAsCsUsC-3′ (SEQ ID NO: 1); where “s” depicts a phosphothioate linkage) complexed with the cationic lipid “LyoVec” to protect the RNA from degradation and enhance is uptake by immune cells. Such ssRNA can be purchased from InvivoGen (San Diego, Calif.).

The TLR agonism for a particular compound may be assessed in any suitable manner. For example, assays and recombinant cell lines suitable for detecting TLR agonism of test compounds are described, for example, in U.S. Patent Publication Nos. US2004/0014779, US2004/0132079, US2004/0162309, and US2004/0197865, the entire contents of which are incorporated herein by reference.

Regardless of the particular assay employed, a compound can be identified as an agonist of a particular TLR if performing the assay with a compound results in at least a certain threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Moreover, a compound may be identified as “selective” if it induces activity of one TLR when administered at a concentration significantly lower than necessary to induce activity of other TLRs. A significant degree may be, for example, inducing activity mediated by one TLR (e.g., TLR8) when administered at half the concentration necessary to induce activity through another TLR (e.g., TLR7). Examples of TLR8-selective compounds include 2-propylthiazolo[4,5-c]quinolin-4-amine, 2-propylthiazolo[4,5-c]quinoline-4,8-diamine, and 2-butylthiazolo[4,5-c][1,5]naphthyridin-4-amine. Some TLR8-selective compounds such as, for example, N-{3-[(4-amino-2-propyl[1,3]thiazolo[4,5-c]quinolin-7-yl)oxy]propyl}-5-(dimethylamino)naphthalene-1-sulfonamide and tert-butyl 2-[(4-amino-2-propyl[1,3]thiazolo[4,5-c]quinolin-7-yl)oxy]ethylcarbamate can induce TLR8 activity at a concentration between about one half to about one-fifth (i.e., at about a two-fold to about a five-fold dilution) of that necessary to induce TLR7-mediated activity. Other TLR8-selective compounds such as, for example, 2-(1-methylethyl)thiazolo[4,5-c]quinolin-4-amine; 2-(2-methylpropyl)thiazolo[4,5-c]quinolin-4-amine; 8-methyl-2-propylthiazolo[4,5-c]quinolin-4-amine; 7-fluoro-2-propylthiazolo[4,5-c]quinolin-4-amine; 2-propylthiazolo[4,5-c[1,5]naphthyridin-4-amine; N-[3-(4-amino-2-propylthiazolo[4,5-c]quinolin-7-yl)phenyl]methanesulfonamide; tert-butyl 3-[(4-amino-2-propyl[1,3]thiazolo[4,5-c]quinolin-7-yl)oxy]propylcarbamate; N-{6-[(4-amino-2-propyl[1,3]thiazolo[4,5-c]quinolin-7-yl)oxy]hexyl}methanesulfonamide; tert-butyl 2-{2-[(4-amino-2-propyl[1,3]thiazolo[4,5-c]quinolin-7-yl)oxy]ethoxy}ethylcarbamate; 7-[2-(2-chloroethoxy)ethoxy]-2-propyl[1,3]thiazolo[4,5-c]quinolin-4-amine can induce TLR8 activity at a concentration less than one-fifth (i.e., at a dilution greater than five-fold) of that necessary to induce TLR7-mediated activity.

The above-identified TLR8-selective compounds are described in, for example, U.S. Pat. Nos. 6,110,929; 6,627,638; 6,440,992; U.S. Patent Publication No. 2005/0021334; and U.S. Pat. Ser. No. 60/651585, the entire contents of which are incorporated herein by reference.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

The screening assays used to identify TLR8 agonists can have any of a number of possible readout systems based upon either the TLR8 signaling pathway or other assays suitable for assaying TLR signaling activity. In one preferred embodiment, the readout for the screening assay is based on the use of native genes or, alternatively, cotransfected or otherwise co-introduced reporter gene constructs which are responsive to the TLR signal transduction pathway involving MyD88, TRAF6, p38, and/or ERK (Hacker H et al., EMBO J 18:6973-6982 (1999)). These pathways activate kinases including kappa B kinase complex and c-Jun N-terminal kinases. Thus reporter genes and reporter gene constructs particularly useful for the assays can include a reporter gene operatively linked to a promoter sensitive to NF-kappa B. Examples of such promoters include, without limitation, those for NF-kappa B, IL-1beta, IL-6, IL-8, IL-12 p40, CD80, CD86, and TNF-α. The reporter gene operatively linked to the TLR8-sensitive promoter can include, without limitation, an enzyme (e.g., luciferase, alkaline phosphatase, beta-galactosidase, chloramphenicol acetyltransferase (CAT), etc.), a bioluminescence marker (e.g., green-fluorescent protein (GFP, U.S. Pat. No. 5,491,084), etc.), a surface-expressed molecule (e.g., CD25), and a secreted molecule (e.g., IL-8, IL-12 p40, TNF-α).

In one preferred embodiment the reporter is selected from IL-8, TNF-α, NF-kappa B-luciferase (NF-kappa B-luc; Hacker H et al., EMBO J 18:6973-6982 (1999)), IL-12 p40-luc (Murphy T L et al., Mol Cell Biol 15:5258-5267 (1995)), and TNF-luc (Hacker H et al., EMBO J 18:6973-6982 (1999)). In assays relying on enzyme activity readout, substrate can be supplied as part of the assay, and detection can involve measurement of chemiluminescence, fluorescence, color development, incorporation of radioactive label, drug resistance, or other marker of enzyme activity. For assays relying on surface expression of a molecule, detection can be accomplished using flow cytometry analysis or functional assays. Secreted molecules can be assayed using enzyme-linked immunosorbent assay (ELISA) or bioassays. Many such readout systems are well known in the art and are commercially available.

Another source of agonists is combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods, such as the methods described herein.

The term “peptide”, as used herein, refers to a compound consisting of from about two to about ninety amino acid residues wherein the amino group of one amino acid is linked to the carboxyl group of another amino acid by a peptide bond.

A peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). A “peptide” can comprise any suitable L-and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, omithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

Peptides can be synthesized and assembled into libraries comprising a few to many discrete molecular species. Such libraries can be prepared using well-known methods of combinatorial chemistry, and can be screened as described herein or using other suitable methods to determine if the library comprises peptides which can activate TLR8 function. Such peptide agonists can then be isolated by suitable means.

The term “peptidomimetic”, as used herein, refers to molecules which are not polypeptides, but which mimic aspects of their structures. For example, polysaccharides can be prepared that have the same functional groups as peptides which can activate TLR8. Peptidomimetics can be designed, for example, by establishing the three dimensional structure of a peptide agent in the environment in which it is bound or will bind to TLR8. The peptidomimetic comprises at least two components, the binding moiety or moieties and the backbone or supporting structure. These compounds can be manufactured by known methods. For example, a polyester peptidomimetic can be prepared by substituting a hydroxyl group for the corresponding a-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the desired chemical structure of the peptidomimetic.

Peptidomimetics can be synthesized and assembled into libraries comprising a few to many discrete molecular species. Such libraries can be prepared using well known methods of combinatorial chemistry, and can be screened as described herein to determine if the library comprises one or more peptidomimetics which activate TLR function. Such peptidomimetic agonists can then be isolated by suitable methods.

Antibodies can also be screened for their ability to activate TLR8 and used in methods of the invention. As used herein, the term “antibody” encompasses polyclonal or monoclonal antibodies as well as functional fragments of antibodies, including fragments of chimeric, human, humanized, primatized, veneered or single-chain antibodies. Functional fragments include antigen-binding fragments which bind to TLR8. For example, antibody fragments capable of binding to TLR8 or portions thereof, including, but not limited to Fv, Fab, Fab′ and F (ab′) 2 fragments can be used. Such fragments can be produced by enzymatic cleavage or by recombinant techniques. For example, papain or pepsin cleavage can generate Fab or F (ab′) 2 fragments, respectively. Other proteases with the requisite substrate specificity can also be used to generate Fab or F (ab′) 2 fragments. Antibodies can also be produced in a variety of truncated forms using antibody genes in which one or more stop codons have been introduced upstream of the natural stop site. For example, a chimeric gene encoding a F (ab′) 2 heavy chain portion can be designed to include DNA sequences encoding the CH, domain and hinge region of the heavy chain.

The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1 and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19 (9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies which are specific for mammalian (e.g., human) TLR8 can be raised against an appropriate immunogen, such as isolated and/or recombinant human TLR8 or portions thereof (including synthetic molecules, such as synthetic peptides).

Agonists of TLR8 useful in the methods of the present invention include IRM compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In certain embodiments, the TLR8 agonist may be one of the following: 4-amino-2-(ethoxymethyl)-α,α-dimethyl-6,7,8,9-tetrahydro-1H-imidazo[4,5-c]quinoline-1-ethanol from Example 91 of U.S. Pat. No. 5,352,784 is an agonist of both TLR7 and TLR8; 2-propylthiazolo[4,5-c]quinolin-4-amine from Example 12 of U.S. Pat. No. 6,110,929; N-(2-{2-[-amino-2-(2-methoxyethyl)-1H-imidazo[4,5-c]quinolin-1-yl ethoxy}ethyl) hexadecanamide which is IRM3 of U.S. Pat. App. No. 2004/0091491; N-{2-[4-amino-2-(ethoxymethyl)-1H-imidazo[4,5-c]quinolin-1-yl]ethyl}methanesulfonamide from U.S. Pat. No. 6,331,539; 2-propyl[1,3]thiazolo[4,5-c]quinolin-4-amine, a predominantly TLR8 agonist cited in WO 04/091500; 4-amino-α,α-dimethyl-2-ethoxymethyl-1H-imidazo[4,5-c]quinolin-1-ethanol, the synthesis of which is described in Example 99 of U.S. Pat. No. 5,389,640; N-{4-[4-amino-2-(2-methoxyethyl)-1H-imidazo[4,5-c]quinolin-1-yl]butyl}quinoline-3-carboxamide described in Example 182 of U.S. Pat. No. 2003/0144283; N-{4-[4-amino-2-(2-methoxyethyl)-1H-imidazo[4,5-c]quinolin-1-yl]butyl}quinoxaline-2-carboxamide described in Example 183 of U.S. Pat. No. 2003/0144283; N-[4-(4-amino-2-propyl-1H-imidazo[4,5-c]quinolin-1-yl)butyl]morpholine-4-carboxamide described in U.S. Pat. No. 6,541,485; 2-propylthiazolo[4,5-c]quinoli-n-4-amine described in Example 12 of U.S. Pat. No. 6,110,929; N¹-[2-(4-amino-2-butyl-1H-imidazo[4,5-c[1,5]naphthyridin-1-yl)ethyl]-2-amino-4-methylpentanamide described in Example 102 of U.S. Pat. No. 6,194,425; N¹-[4-(4-amino-1H-imidazo[4,5-c]quinolin-1-yl)butyl]-2-phenoxybenzamide described in Example 14 of U.S. Pat. No. 6,451,810; N¹-[2-(4-amino-2-butyl-1H-imidazo[4,5-c]quinolin-1-yl)ethyl]-1-propanesulfonamide described in Example 17 of U.S. Pat. No. 6,331,539; N-{2-[2-(4-amino-2-ethyl-1H-imidazo[4,5-c]quinolin-1-yl)ethoxy]ethyl}-N′-phenylurea described in Example 50 of U.S. Pat. App. No. 2003/0130518; 1-{4-[(3,5-dichlorophenyl)thio]butyl}-2-ethyl-1H-imidazo[4,5-c]quinolin-4-amine described in Example 44 of U.S. Pat. App. No. 2003/0100764; N-{2-[4-amino-2-(ethoxymethyl)-1H-imidazo[4,5-c]quinolin-1-yl]ethyl}-N′-(3-cyanophenyl)urea described in WO 00/76518; 4-amino-2-ethoxymethyl-α,α-dimethyl-6,7,8,9-tetrahydro-1H-imidazo[4,5-c]quinoline-1-ethanol described in Example 91 of U.S. Pat. No. 5,352,784; 4-amino-α,α-dimethyl-2-methoxye-thyl-1H-imidazo[4,5-c]quinoline-1-ethanol described in Example 111 of U.S. Pat. No. 5,389,640; 4-amino-2-butyl-α,α,6,7-tetramethyl-1H-imidazo[4,5-c]pyridine-1-ethanol described in Example 52 of U.S. Pat. No. 5,494,916; N-(2-{2-[4-amino-2-(2-methoxyethyl)-1H-imidazo[4,5-c]quinolin-1-yl)ethoxy}ethyl)-N′-phenylurea described in Example 1 in WO 02/46191; 1-{4-[(3,5-dichlorophenyl)sulfonyl]butyl}-2-ethyl-1H-imidazo[4,5-c]quinolin-4-amine described in Example 46 of U.S. Pat. App. No. 2003/0100764; N-{2-[4-amino-2-(2-methoxyethy-1)-1H-imidazo[4,5-c]quinolin-1-yl]ethyl}-N′-sec-butylthiourea described in WO 00/76518; and N-{2-[4-amino-2-(2-methoxyethyl)-6,7,-8,9-tetrahydro-1H-imidazo[4,5-c]quinolin-1-yl]-1,1-dimethylethyl}methanesulfonamide U.S. Pat. No. 6,331,539.

In certain embodiments, the TLR8 agonist is a TLR8-selective small molecule immune response modifier (IRM) compound. Such compounds include, for example, thiazoloquinoline amines including but not limited to 2-propylthiazolo[4,5-c]quinolin-4-amine, 2-propylthiazolo[4,5-c]quinoline-4,8-diamine, 2-butylthiazolo[4,5-c[1,5]naphthyridin-4-amine, N-{3-[(4-amino-2-propyl[1,3]thiazolo[4,5-c]quinolin-7-yl)oxy]propyl}-5-(dimethylamino)naphthalene-1-sulfonamide, tert-butyl 2-[(4-amino-2-propyl[1,3]thiazolo[4,5-c]quinolin-7-yl)oxy]ethylcarbamate, 2-(1-methylethyl)thiazolo[4,5-c]quinolin-4-amine, 2-(2-methylpropyl)thiazolo[4,5-c]quinolin-4-amine, 8-methyl-2-propylthiazolo[4,5-c]quinolin-4-amine, 7-fluoro-2-propylthiazolo[4,5-c]quinolin-4-amine, 2-propylthiazolo[4,5-c][1,5]naphthyridin-4-amine, N-[3-(4-amino-2-propylthiazolo[4,5-c]quinolin-7-yl)phenyl]methanesulfonamide, tert-butyl 3-[(4-amino-2-propyl[1,3]thiazolo[4,5-c]quinolin-7-yl)oxy]propylcarbamate, N-{6-[(4-amino-2-propyl[1,3]thiazolo[4,5-c]quinolin-7-yl)oxy]hexyl}methanesulfonamide, tert-butyl 2-{2-[(4-amino-2-propyl[1,3]thiazolo[4,5-c]quinolin-7-yl)oxy]ethoxy}ethylcarbamate, and 7-[2-(2-chloroethoxy)ethoxy]-2-propyl[1,3]thiazolo[4,5-c]quinolin-4-amine, which are described in, for example, U.S. Pat. Nos. 6,110,929; 6,627,638; 6,440,992; U.S. Patent Publication No. 2005/0021334; and U.S. Patent Ser. No. 60/651585.

### Pharmaceutically Acceptable Formulations

The compounds or agents of the present invention can be contained in pharmaceutically acceptable formulations. Such a pharmaceutically acceptable formulation may include a pharmaceutically acceptable carrier(s) and/or excipient(s). As used herein, “pharmaceutically acceptable carrier” includes any and all solvents, dispersion media, coatings, antibacterial and anti fungal agents, isotonic and absorption delaying agents, and the like that are physiologically compatible. For example, the carrier can be suitable for injection into the cerebrospinal fluid. Excipients include pharmaceutically acceptable stabilizers. The present invention pertains to any pharmaceutically acceptable formulations, including synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes.

In some embodiments, the TLR8 agonist may be administered to a subject in a formulation that includes, for example, from about 0.001% to about 10% TLR8 agonist (unless otherwise indicated, all percentages provided herein are weight/weight with respect to the total formulation), although in some embodiments the TLR8 agonist may be administered using a formulation that provides the TLR8 agonist in a concentration outside this range. In certain embodiments, the formulation may include from about 0.01% to about 1% TLR8 agonist such as, for example, from about 0.1% to about 0.5% TLR8 agonist.

In one embodiment, the pharmaceutically acceptable formulations comprise a polymeric matrix. The terms “polymer” or “polymeric” are art-recognized and include a structural framework comprised of repeating monomer units which is capable of delivering an agent such that treatment of a targeted condition occurs. The terms also include co-polymers and homopolymers such as synthetic or naturally occurring. Linear polymers, branched polymers, and cross-linked polymers are also meant to be included.

For example, polymeric materials suitable for forming the pharmaceutically acceptable formulation employed in the present invention, include naturally derived polymers such as albumin, alginate, cellulose derivatives, collagen, fibrin, gelatin, and polysaccharides, as well as synthetic polymers such as polyesters (PLA, PLGA), polyethylene glycol, poloxomers, polyanhydrides, and pluronics. These polymers are biocompatible and biodegradable without producing any toxic byproducts of degradation, and they possess the ability to modify the manner and duration of the active compound release by manipulating the polymer's kinetic characteristics. As used herein, the term “biodegradable” means that the polymer will degrade over time by the action of enzymes, by hydrolytic action and/or by other similar mechanisms in the body of the subject. As used herein, the term “biocompatible” means that the polymer is compatible with a living tissue or a living organism by not being toxic or injurious and by not causing an immunological rejection. Polymers can be prepared using methods known in the art.

The polymeric formulations can be formed by dispersion of the active compound within liquefied polymer, as described in U.S. Pat. No. 4,883,666, the teachings of which are incorporated herein by reference or by such methods as bulk polymerization, interfacial polymerization, solution polymerization and ring polymerization as described in Odian G., Principles of Polymerization and ring opening polymerization, 2nd ed., John Wiley & Sons, New York, 1981, the contents of which are incorporated herein by reference. The properties and characteristics of the formulations are controlled by varying such parameters as the reaction temperature, concentrations of polymer and the active compound, the types of solvent used, and reaction times.

The active therapeutic compound can be encapsulated in one or more pharmaceutically acceptable polymers, to form a microcapsule, microsphere, or microparticle, terms used herein interchangeably. Microcapsules, microspheres, and microparticles are conventionally free-flowing powders consisting of spherical particles of 2 millimeters or less in diameter, usually 500 microns or less in diameter. Particles less than 1 micron are conventionally referred to as nanocapsules, nanoparticles or nanospheres. For the most part, the difference between a microcapsule and a nanocapsule, a microsphere and a nanosphere, or microparticle and nanoparticle is size; generally there is little, if any, difference between the internal structure of the two. In one aspect of the present invention, the mean average diameter is less than about 45 μm, preferably less than 20 μm, and more preferably between about 0.1 and 10 μm.

In another embodiment, the pharmaceutically acceptable formulations comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used.

Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing an active compound such variables as the efficiency of active compound encapsulation, labiality of the active compound, homogeneity and size of the resulting population of vesicles, active compound-to-lipid ratio, permeability, instability of the preparation, and pharmaceutical acceptability of the formulation should be considered.

Prior to introduction, the formulations can be sterilized, by any of the numerous available techniques of the art, such as with gamma radiation or electron beam sterilization.

Ophthalmic products for topical use may be packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquatemium-1, or other agents known to those skilled in the art. Such preservatives are typically employed at a level of from 0.001 to 1.0% weight/volume (“% w/v”). Such preparations may be packaged in dropper bottles or tubes suitable for safe administration to the eye, along with instructions for use.

### Administration of the Pharmaceutically Acceptable Formulations to a Patient

When the agents or compounds are delivered to a patient, they can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. Administration can be local or systemic as indicated. Agents can also be delivered using viral vectors, which are well known to those skilled in the art.

Both local and systemic administration are contemplated by the invention. Desirable features of local administration include achieving effective local concentrations of the active compound as well as avoiding adverse side effects from systemic administration of the active compound.

The pharmaceutically acceptable formulations can be suspended in aqueous vehicles and introduced through conventional hypodermic needles or using infusion pumps.

In one embodiment, the active compound formulation described herein is co-administered with another therapeutic agent or vaccine. The TLR8 agonist can be administered before, concurrently with, or after administration of the additional agent.

The amount of agent administered to the individual will depend on the characteristics of the individual, such as general health, age, sex, body weight and tolerance to drugs as well as the degree, severity and type of rejection. The skilled artisan will be able to determine appropriate dosages depending on these and other factors. Typically, an effective amount can range from about 0.1 m/kg per day to about 100 m/kg per day.

Antibodies and antigen-binding fragments thereof, particularly human, humanized and chimeric antibodies and antigen-binding fragments can often be administered less frequently than other types of therapeutics. For example, an effective amount of such an antibody can range from about 0.01 m/kg to about 5 or 10 m/kg administered daily, weekly, biweekly, monthly or less frequently.

In one preferred embodiment, a TLR8 agonist is used as an adjuvant to enhance/induce the immune response of a newborn to an antigen of a vaccine formulation. The agonists of the invention can be used with antigens derived from any pathogen, e.g. any bacteria, fungus, parasite, or virus, provided the antigen does not get destroyed or denatured. Examples of some antigens, and certainly not by way of limitation, are Erysipelothrix rhusiopathiae antigens, Bordetella bronchiseptica antigens, antigens of toxigenic strains of Pasteurella multocida, antigens of Escherichia coli strains that cause neonatal diarrhea, Actinobacillus pleuropneumoniae antigens, Pasteurella haemiolytica antigens, or any combination of the above. Adjuvants of the invention are also useful in vaccine compositions that contain an antigen described in U.S. Pat. Nos. 5,616,328 and 5,084,269.

Acute infections that can be treated by methods of the invention include any viral, fugal, parasitic, or bacterial infection caused by any pathogen. Some pathogens include, for example, Group B streptococcus, Bordetella spp., Listeria monocytogenes, Bacillus anthracis, S. pneumoniae, N. meninigiditis, hepatitis, measles, poliovirus, human immunodeficiency virus, influenza virus, parainfluenza virus, respiratory syncytial virus, herpes simplex virus, M. tuberculosis, Leishmania, Schistosomes, Trypanosomes, toxoplasma, pneumocystis and Candida spp., Cryptococcus, Coccidiodes, Aspergillus spp., as well as others.

In one embodiment, the TLR8 immunomodulatory agonist of the invention is used in a vaccine for immunotherapy of cancer in a newborn. Such cancer vaccines are known to those in the art.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLES

### Example I

Peripheral blood was collected from healthy adult volunteers (N=26 individual volunteers; mean age 27 years; 45% male, 55% female) and newborn cord blood (N=63; mean gestational age 39 weeks; 43% male, 57% female) collected immediately after cesarean section delivery (epidural anesthesia) of the placenta or from the umbilical cord immediately after vaginal birth but prior to delivery of the placenta. Births at which antibiotics were administered during labor and/or delivery, and births to HIV-positive mothers were excluded. Human experimentation guidelines of the US Department of Health and Human Services and the Brigham & Women's Hospital were observed, following protocols approved by the local Institutional Review Board. Blood was anticoagulated with 129 mM sodium citrate (Becton Dickinson, Franklin Lakes, N.J.). Hemocytes were collected by centrifugation of blood, followed by washing three times with Hank's Balanced Salt Solution (HBSS) buffer without magnesium or calcium (Gibco BRL, Grand Island, N.Y.) and then resuspension in either autologous or heterologous 100% plasma.

TLR ligands included the synthetic triacylated BLP (tBLP) Pam3-CSSNA (Bachem Bioscience, King of Prussia, PA) corresponding to the N-terminus of a BLP from E. coli B/r (Biesert et al. 1987. Eur J Biochem 162:651), the synthetic diacylated BLP macrophage-activating lipopeptide-2 (MALP; S-(2,3-bisAcyloxypropyl)-cysteine-GNNDESNISFKEK; Alexis Biochemicals, Lausen, Switzerland) from Mycoplasma fermentans (Muhlradt et al. 1997. J Exp Med. 185:1951), ultrapure Re 595 LPS from Salmonella minnesota (List Biologicals, Campbell, Calif.), and the IRM compounds imiquimod (3M Pharmaceuticals, Northridge, Calif.), and resiquimod (InvivoGen, San Diego, Calif.). Specificity of individual TLR ligands for their cognate receptors was confirmed using either NF-κB luciferase reporter and TLR co-transfected Human Embryonic Kidney (HEK) 293 cells or a neutralizing mAb to TLR2 (Levy, O. et al. 2003. Infect Immun 71:6344), as previously described.

Heparinized blood was layered onto Ficoll-Hypaque gradients, the peripheral blood mononuclear cell (PBMC) layer collected, and subjected to hypotonic lysis to remove red blood cells. Monocytes were isolated from PBMC by positive selection using magnetic microbeads coupled to an anti-CD14 mAb according to the manufacturer's instructions (Miltenyi Biotec, Auburn, Calif.) and stimulated in the presence of 100% autologous serum.

After incubation of TLR ligands in blood or monocyte suspensions for 5 hours at 37° C. with end-over-end rotation, samples were diluted with five volumes of ice-cold RPMI medium (Gibco BRL) and centrifuged at 1,000×g at 4° C. for 5 minutes. The supernatant was recovered and stored at −20° C. until assay of TNF-α by ELISA (R&D Systems, Minneapolis, Minn.).

Both purified recombinant human sCD14 and sCD14 ELISA for measurement of concentrations in citrated newborn and adult plasma or serum were from R&D Systems. For experiments in which sCD14 was replenished in newborn cord blood, either 500 or 1,000 ng of pure sCD14 were added per mL of whole blood.

Total RNA was isolated using a silica-gel-based membrane (RNeasy, Qiagen, Valencia, Calif.) and treated with DNase (Qiagen) to avoid contamination with genomic DNA. Random-primed cDNA was prepared using a reverse transcription kit per the manufacturer's instructions (Clontech, Palo Alto, Calif.). Taqman PCR was performed to measure the relative mRNA levels of the TLR or TLR-related molecules as previously described (Zarember, K. A. et al. 2002.[erratum appears in J Immunol Jul. 15, 2002;169(2): 1136]. J Immunol. 168:554.), except for TIRAP primers: forward 5′-CCTGAGCTCCGATTCATGT-3′ (SEQ ID NO: 2), probe FAM-5′-CCCTGATGGTGGCTTTCGTCAA-3′-TAMRA (SEQ ID NO: 3), and reverse 5′-CGCATGACAGCTTCTTTGA-3′ (SEQ ID NO: 4). Bonferroni's method of statistical analysis for multiple comparisons was employed to compare relative mRNA expression in newborn and adult monocytes. Human TNF-α mRNA was measured using specific PreDeveloped Assay Reagents (Applied Biosystems, Foster City, Calif.).

Total cellular TLR2 content of purified monocytes or control THP-1 cells was measured using a TLR2 ELISA as follows. Maxisorp plates were coated with 0.25 μg/well mAb #2420 in PBS overnight at 4° C. After a brief wash with PBS, plates were incubated with shaking at room temperature in blocking buffer (150 mM NaCl, 10 mM HEPES pH 7.2, 0.25% BSA, 0.05% Tween-20, 1 mM EDTA, 0.05% NaN3). Cel lysates wert prepared in 1% Triton-X-100, 150 mM NaCl, 10% glycerol, 2 mM EDTA, 25 mM HEPES, pH 7.2 supplemented with a standard protease inhibitor cocktail. 100 μl fresh blocking buffer was added to each well followed by up to 100 μl of sample (balance block solution) and incubated at 4° C. with shaking overnight. After washing 3× with PBS, each well was incubated with 200 μl mAb #2392:HRPO conjugate for 1 hour then washed 3× with PBS/0.05% Tween-20, once with PBS, developed with 100 μl ABTS solution (Calbiochem, San Diego, Calif.), stopped with 1M H2SO4 (100 μl) and measured at 405 mn. TLR2 ELISA specificity was confirmed by testing lysates prepared from HEK293 cells transiently transfected with plasmids encoding tagged versions of all human TLRs (1-10), with only TLR2 expressing cells producing a measurable signal.

TLR ligands were added to citrated blood at a final concentration of 100 ng/mL (LPS) or 10 μg/mL (tBLP). In some experiments, 10 μg/mL of brefeldin A (Sigma-Aldrich, St. Louis, Mo.) was added to the blood before the TLR ligand to inhibit TNF-α secretion and enhance detection of intracellular TNF-α. Quantitative surface expression of TLRs and CD14 was measured using phycoerythrin (PE)-conjugated mAbs (eBiosciences, San Diego, Calif.) incubated at RT for 30 minutes. To identify monocytes, samples stained for TLRs with PE-conjugated mAb's were co-stained for CD14 using a FITC-conjugated CD14 mAb (eBiosciences). After red blood cell lysis using 1× FACSLyse solution and permeabilization using 1× FACSPerm2 Solution (BD Biosciences), samples were washed with 1× PBS/0.5% HSA. To determine which blood leukocytes synthesize TNF-α in response to TLR ligands, cells were stained for intracellular TNF-α according to the manufacturer's protocol (BD Biosciences). TNF-α was stained with a PE-conjugated TNF-α mAb using murine IgG1 as control and monocytes were identified using FITC-conjugated CD14 mAb. Phosphorylated p38 MAP kinase was stained in permeabilized cells using a PE-conjugated phospho-specific (pT180/pY182) p38 mouse IgG1 mAb (clone 36, BD Biosciences). Flow cytometry was performed using a MoFlo cytometer (DakoCytomation, Fort Collins, Colo.) with a 488-nm laser. Data were analyzed with Summit v 7.19 software (DakoCytomation). To compare intracellular TNF-α production by monocytes in newborn and adult blood, a TNF-α production index was calculated based on the mean fluorescence intensity (MFI): (% of total leukocytes that are monocytes)×(% monocytes that are TNF-α-positive)×(MFI of TNF-α positive monocytes/MFI of monocytes stained with an isotype control antibody).

### Example II

TLR ligand-induced TNF-α release in whole human blood ex vivo as described above in Example 1. The single stranded ribonucleic acid (ssRNA) tested in this example was ssRNA40/LyoVec purchased from InvivoGen (San Diego, Calif.) comprised of single-stranded GU-rich oligonucleotide (5′-GsCsCsCsGsUsCsUsGsUsUsGsUsGsUsGsAsCsUsC-3′ (SEQ ID NO: 1); where “s” depicts a phosphothioate linkage complexed with the cationic lipid LyoVec that protects the RNA from degradation and enhance is uptake by immune cells. The guanosine analog loxoribine (TLR7 ligand) was purchased from InvivoGen.

TABLE I
TLR Agonists Used in Example II

| Agonist | TLR | Derivation | Source | Comment | Ref. |
|---|---|---|---|---|---|
| MALP | 2/6 | Mycoplasma fermentans | Alexis | | [25] |
| | | | Biochemicals | | |
| LPS | 4 | Salmonella minnesota | List Biological | Ultra-pure | [26] |
| | | R595 (Re) | Laboratories, | | |
| | | | Inc. | | |
| Loxoribine | 7 | Guanosine analog | Invivo Gen, Inc. | | [27] |
| Imiquimod | 7 | imidazoquinoline | Sequoia, U.K. | Aldara antiviral | [19] |
| | | | | cream | |
| IRM3 | 7/8 | thiazoloquinoline amine | 3M Pharm. | 4-amino-2- | [23] |
| | | | | (ethoxymethyl)-α,α- | |
| | | | | dimethyl-6,7,8,9- | |
| | | | | tetrahydro-1H- | |
| | | | | imidazo[4,5- | |
| | | | | c]quinoline-1-ethanol | |
| Resiquimod | 7/8 | imidazoquinoline | InvivoGen | Resiquimod | [24] |
| IRM2 | 8 | tetrahydroimidazoquinoline | 3M Pharm. | 2-propylthiazolo[4,5- | [23] |
| | | amine | | c]quinolin-4-amine | |
| ssRNA | 8 | Synthetic poly-Uridine | InVivoGen | complexed to | [20] |
| poly U | | sequence | | cationic lipid to | |
| | | | | facilitate uptake | |
| ssRNA40 | 8 | Synthetic GU-rich | InvivoGen | complexed to | [20] |
| | | sequence based upon U5 | | cationic lipid to | |
| | | region of HIV | | facilitate uptake | |

### Example III

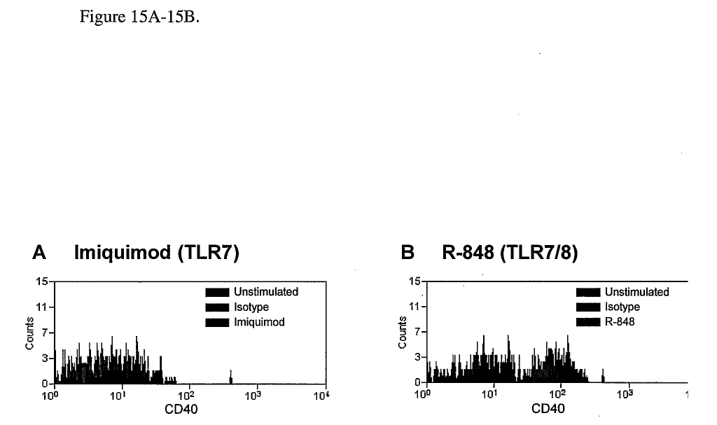

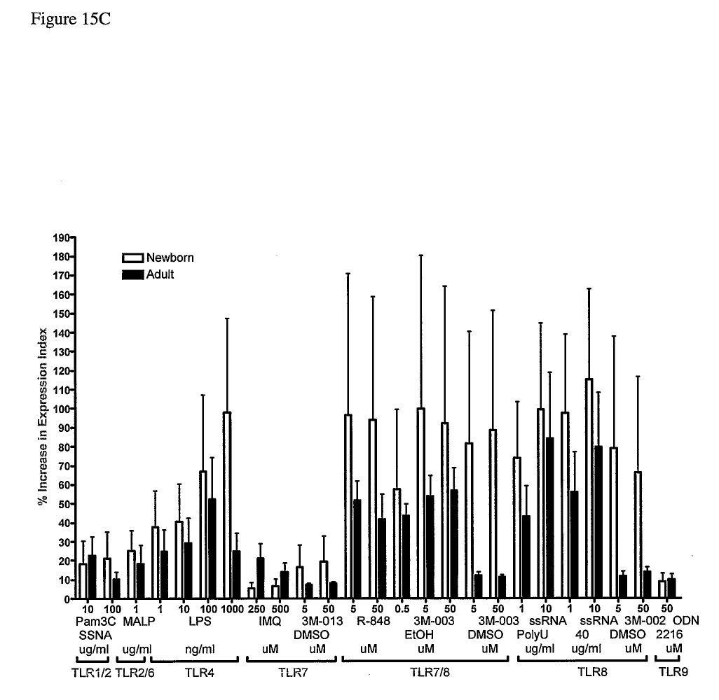

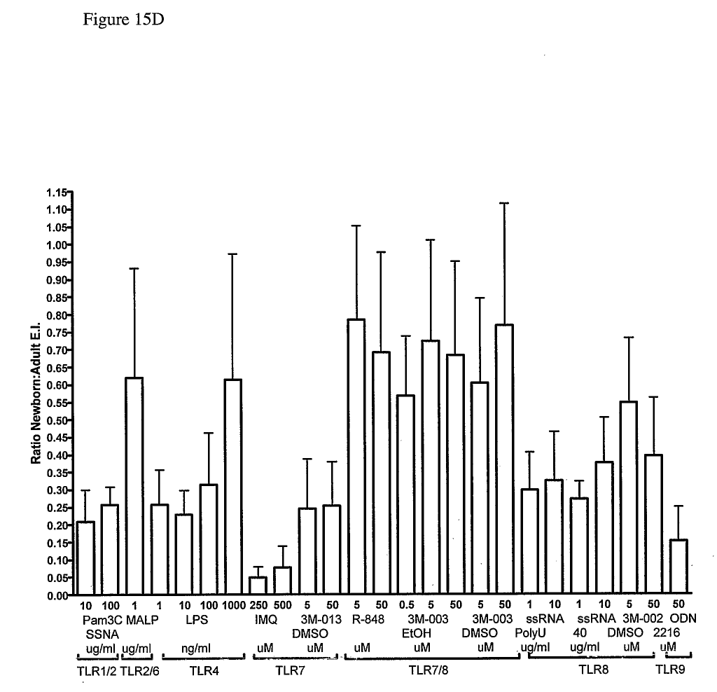

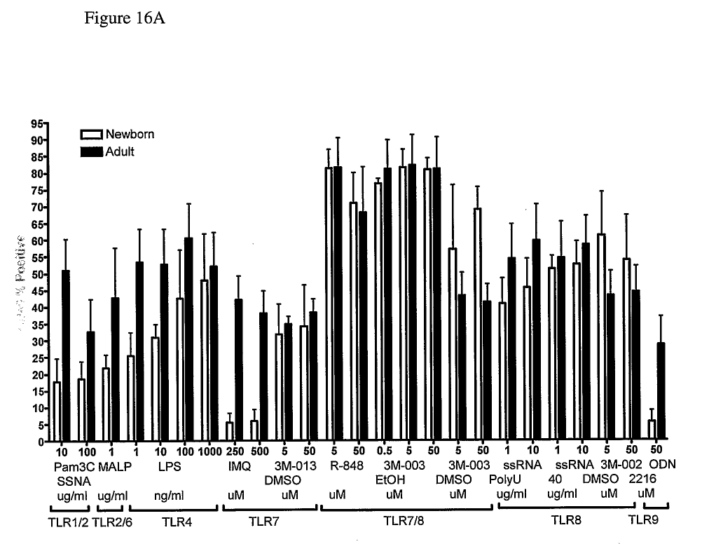

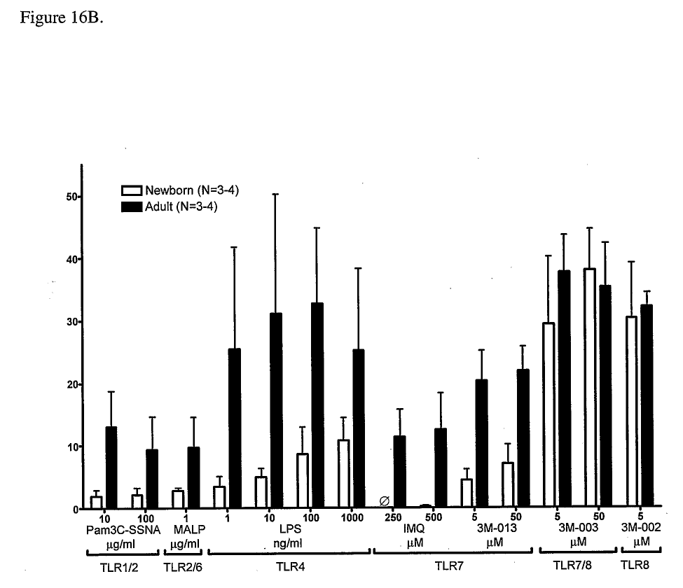

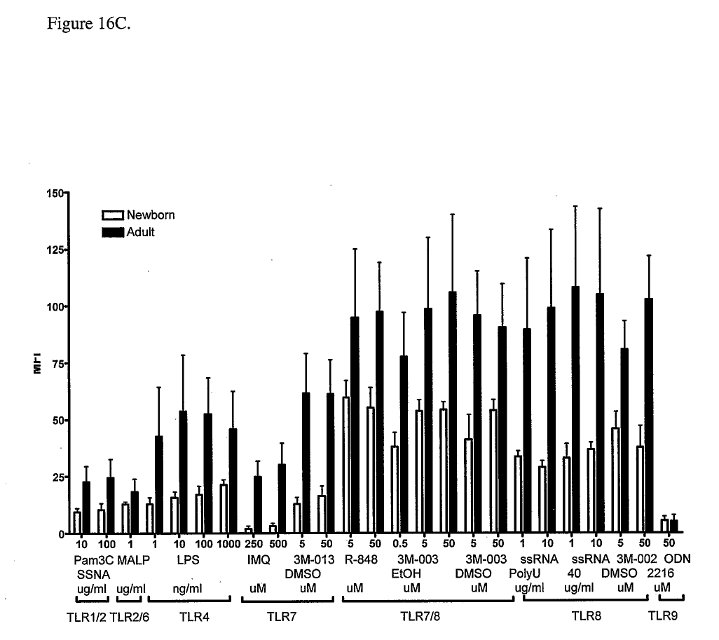

CD40 expression on mDCs was studied in whole newborn cord blood, in comparison to those of adult peripheral blood, using four-color flow cytometry (BD Biosciences). mDCs were identified as lineage 1-/HLA-DR+/CD11c+ cells. Upregulation of surface CD40 expression was measured using a phycoerythrin-conjugated anti-CD40 mAb. Data for the effects of imiquimod (TLR7) and resiquimod (TLR 7/8) are shown in FIGS. 15A-15D.

### Example IV

Newborn (1 day old) and adult (6-8 week old) Balb/c mice (obtained from The Jackson Laboratory) may be immunized subcutaneously with OVA in the absence or presence of a TLR7/8 agonist (selected from those in Table I based upon consistent and potent stimulatory activity of neonatal APCs as measured in example 5) or the TLR4 agonist LPS, neonatal responses to which are often impaired. Antigen-specific CD4+ and CD8+T cells as well as antibody responses may be measured.

TLR agonists may be injected at day zero with OVA. Splenic and lymph node T cells may be studied at multiple time-points after immunization. Blood may be collected to prepare serum that may be tested for OVA-specific antibodies. T cell proliferation assays may be performed at 7 days post-immunization and antibodies may be measured at 0, 7, 14, and 21 days post-immunization (robust antibody production by day 14 may occur). Specific protocols are described below:

Immunization. Groups of five neonatal (1 day old; derived from pregnant female mice; The Jackson Laboratory) and five adult (6-8 week old) BALB/c mice may be injected subcutaneously (s.c.) at the base of tail with a total of 100 μL of fluid containing one of the following stimuli: 1) OVA (100 μg/mouse) (Grade III; Sigma, St Louis, Mo.) in 100 μl of phosphate-buffered saline (PBS), 2) OVA with LPS, 3) OVA with TLR8 (±7) agonist. Seven days later, the mice may be sacrificed (according to Institutional and IRB-approved standards) and the draining lymph nodes (LN) harvested for preparation of OVA-specific T-cell lines and clones. Interpretable and consistent results from the first experiment with 5 mice in each group prove, indicate immunizing another 10 mice in each group (i.e., total of 15 mice per group).

Preparation of splenocytes and lymph node (LN) cells. For preparation of LN cells, draining LNs may be removed from mice 7 days after immunization witn OVA. Single-cell suspensions may be prepared by gentle grinding of LNs on stainless steel sieves in PBS. After washing with PBS, the cells may be counted and resuspended in culture medium at an appropriate concentration. To prepare a single-cell suspension of spleen cells (splenocytes), spleens may be removed from mice and gently ground on stainless steel sieves in 5 ml of PBS. After centrifugation at 1500 g for 5 min and erythrocyte lysis (lysis buffer; Sigma), remaining cells (including T and B lymphocytes, macrophages and DCs) may be washed, counted and resuspended in culture medium at an appropriate concentration.

Cytokine analysis. Splenocytes (5×10⁶) from mice following immunization may be incubated in wells of 24-well Costar plates in the presence of 250 μg/mL OVA (or buffer control) for 3 days at 37° C./5% CO₂. Secretion of the Th1-polarizing polarizing cytokines IL-2, IL-4 and IFN-γ into the culture supernatant may be quantified by ELISA (R&D Systems).

Proliferation assays. Freshly prepared draining LN cells and splenocytes (4×10⁵) from mice post-immunization may be incubated in 96-well flat-bottomed plates (Nunc, Roskilde, Denmark) with irradiated stimulatory T cells or OVA, at different concentrations in a total volume of 200 μl of R10. Cultures may be incubated at 37° in 5% CO2 for 4 days. During the last 8 hr of incubation, [³H]thymidine ([³H]TdR, 0.5 μCi) may be added to each well. Con A may be used as a positive and medium alone as a negative control. The cells may be harvested onto fiber-glass filters and radioactivity measured using a MicroBeta Trilux LSC counter (EG & G Wallac, Turku, Finland).

Antibody measurement. OVA-specific antibodies may be measured by ELISA. 96-well microplates may be coated with OVA (150 μg/well) in carbonate buffer (pH 9.6) and incubated overnight at 4° C. Serum samples may be diluted in a total volume of 200 μL PBS at 37° C. for 1 h, followed by isotype specific HRP-conjugated rabbit anti-mouse Abs (Zymed, San Francisco), and substrate: o-phenylenediamine in citrate buffer (pH 5.0) and 0.02% H₂O₂. Absorbance may be read at 490 nm. Specific OVA isotype titers may be calculated by the product of absorbance and the reciprocal of the sera dilution from an average of two points in the linear portion of the dilution curve. The Th1 -polarizing adjuvant activity of TLR 8 (±7) may be associated with increases in the proportion of anti-OVA antibodies of the IgG2a sub-class.

The references cited throughout the specification are incorporated herein in their entirety by reference.

### REFERENCES

1. Lewis, D. B. and C. B. Wilson, Developmental Immunology and Role of Host Defenses in Fetal and Neonatal Susceptibility to Infection, in Infectious Diseases of the Fetus and Newborn Infant, J. Remington and J. Klein, Editors. 2001, W.B. Saunders: Philadelphia. p. 25-138.
2. Tappero, J. W., et al., Immunogenicity of 2 serogroup B outer-membrane protein meningococcal vaccines: a randomized controlled trial in Chile. [see comment]. JAMA, 1999. 281(16): p. 1520-7.
3. Adkins, B., Development of neonatal Th1/Th2 function. International Reviews of Immunology, 2000. 19 (2-3): p. 157-71.
4. Akira, S. and K. Takeda, Toll-like receptor signaling. Nature Reviews Immunology, 2004. 4: p. 499-511.
5. Levy, O., et al., Selective impairment of Toll-like receptor-mediated innate immunity in human newborns: Neonatal blood plasma reduces monocyte TNF-alpha induction by bacterial lipopeptides, lipopolysaccharide, and imiquimod but preserves response to R-848. J Immunol, 2004. 173: p. 4627-4634.
6. Banchereau, J., et al., Immunobiology of Dendritic Cells. Annual Review of Immunology, 2000. 18: p. 767-811.
7. Steinman, R. M. and K. Inaba, Myeloid dendritic cells. Journal of Leukocyte Biology, 1999. 66(2): p. 205-8.
8. Janeway, C. A., Jr. and R. Medzhitov, Innate immune recognition. Annual Review of Immunology, 2002. 20: p. 197-216.
9. Grewal, I. S. and R. A. Flavell, CD40 and CD154 in cell-mediated immunity. Annual Review of Immunology, 1998. 16: p. 111-35.
10. Quezada, S. A., et al., CD40/CD154 interactions at the interface of tolerance and immunity. Annual Review of Immunology, 2004. 22: p. 307-28.
11. Trinchieri, G., Interleukin-12, in Inflammation: Basic Principles and Clinical Correlates, J. I. Gallin and R. Snyderman, Editors. 1999, Lippincott Williams & Wilkins: Philadelphia. p. 505-512.
12. Evans, J. T., et al., Enhancement of antigen-specific immunity via the TLR4 ligands MPL adjuvant and Ribi.529. Expert Review of Vaccines, 2003. 2(2): p. 219-29.
13. Hunt, D. W., et al., Studies of human cord blood dendritic cells: evidence for functional immaturity. Blood, 1994. 84(12): p. 4333-43.
14. Adkins, B., C. Leclerc, and S. Marshall-Clarke, Neonatal adaptive immunity comes of age. Nature Reviews. Immunology., 2004. 4(7): p. 553-64.
15. Goriely, S., et al., A defect in nucleosome remodeling prevents IL-12(p35) gene transcription in neonatal dendritic cells. Journal of Experimental Medicine, 2004. 199(7): p. 1011-6.
16. Vekemans, J., et al., Neonatal bacillus Calmette-Guerin vaccination induces adult-like IFN-gamma production by CD4+T lymphocytes. European Journal of Immunology, 2001. 31(5): p. 1531-5.
17. Du, X., et al., Three novel mammalian toll-like receptors: gene structure, expression, and evolution. European Cytokine Network., 2000. 11(3): p. 362-71.
18. Chuang, T. H. and R. J. Ulevitch, Cloning and characterization of a sub-family of human toll-like receptors: hTLR7, hTLR8 and hTLR9. European Cytokine Network., 2000. 11(3): p. 372-8.
19. Hemmi, H., et al., Small anti-viral compounds activate immune cells via the TLR7 MyD88-dependent signaling pathway. [comment]. Nature Immunology., 2002. 3(2): p. 196-200.
20. Heil, F., et al., Species-specific recognition of single-stranded RNA via toll-like receptor 7 and 8.[see comment]. Science, 2004. 303(5663): p. 1526-9.
21. Levy, O., et al., Human newborn blood monocytes demonstrate low TNF-alpha but high IL-6 responses to an array of Toll-like receptor ligands: role of a low-molecular weight plasma inhibitor of TNF-alpha release. Journal of Endotoxin Research (Conference Abstracts), 2004. 10(5) p. 370.
22. Ahonen, C. L., et al., Dendritic cell maturation and subsequent enhanced T-cell stimulation induced with the novel synthetic immune response modifier R-848. Cellular Immunology, 1999. 197(1): p. 62-72.
23. Gorden, K. B., et al., Synthetic TLR agonists reveal functional differences between human TLR7 and TLR8. Journal of Immunology, 2005. 174(3): p. 1259-68.
24. Jurk, M., et al., Human TLR7 or TLR8 independently confer responsiveness to the antiviral compound R-848.[comment]. Nature Immunology., 2002. 3(6): p. 499.
25. Galanos, C., et al., MALP-2, a Mycoplasma lipopeptide with classical endotoxic properties: end of an era of LPS monopoly? Journal of Endotoxin Research., 2000. 6(6): p. 471-6.
26. Hoshino, K., et al., Cutting edge: Toll-like receptor 4 (TLR4)-deficient mice are hyporesponsive to lipopolysaccharide: evidence for TLR4 as the Lps gene product. Journal Of Immunology, 1999. 162(7): p. 3749-52.
27. Heil, F., et al., The Toll-like receptor 7 (TLR7)-specific stimulus loxoribine uncovers a strong relationship within the TLR7, 8 and 9 subfamily. European Journal of Immunology, 2003. 33(11): p. 2987-97.
28. Bender, A., et al., Improved methods for the generation of dendritic cells from nonproliferating progenitors in human blood. Journal of Immunological Methods, 1996. 196(2): p. 121-35.
29. Kruisbeek, A. and E. Shevach, Proliferative Assays for T cell Function, in Current Protocols in Immunology, R. Coicio, Editor. 1991, John Wiley & Sons, Inc.: Bethesda, MD. p. 3.12.1-3.12.14.
30. Smith, K., C. Beadling, and E. Jacobson, Interleukin-2, in Inflammation: Basic Principles and Clinical Correlates, J. I. Gallin and R. Snyderman, Editors. 1999, Lippincott Williams & Wilkins: Philadelphia.
31. Thomsen, L. L., et al., Imiquimod and resiquimod in a mouse model: adjuvants for DNA vaccination by particle-mediated immunotherapeutic delivery. Vaccine, 2004. 22(13-14): p. 1799-809.

1. A method for enhancing the immune response of a newborn comprising administering to said newborn an effective amount of a compound or agent that is an agonist of Toll-Like receptor 8 (TLR8).

2. The method of claim 1, wherein the immune response is a Th1 immune response.

3. The method of claim 1, wherein the immune response is an innate immune response.

4. The method of claim 1, wherein the immune response is a local immune response.

5. The method of claim 1, wherein the immune response is a mucosal immune response.

6. The method of claim 1, wherein the immune response is a systemic immune response.

7. The method of claim 1, wherein said agonist is an imidazoquinoline compound.

8. The method of claim 7, wherein the imidazoquinoline compound is 4-amino-α,α-dimethyl-2-ethoxymethyl-1H-imidazo[4,5-c]quinolin-1-ethanol.

9. The method of claim 1, wherein the TLR8 agonist is a tetrahydroimidazoquinoline amine.

10. The method of claim 9, wherein the tetrahydroimidazoquinoline amine is 4-amino-2-(ethoxymethyl)-α,α-dimethyl-6,7,8,9-tetrahydro-1H-imidazo[4,5-c]quinoline-1-ethanol.

11. The method of claim 1, wherein the TLR8 agonist is a thiazoloquinoline amine.

12. The method of claim 11 wherein the thiazoloquinoline amine is 2-propylthiazolo[4,5-c]quinolin-4-amine, 2-propylthiazolo[4,5-c]quinoline-4,8-diamine, 2-butylthiazolo[4,5-c[1,5]naphthyridin-4-amine, N-{3-[(4-amino-2-propyl[1,3]thiazolo[4,5-c]quinolin-7-yl)oxy]propyl}-5-(dimethylamino)naphthalene-1-sulfonamide, tert-butyl 2-[(4-amino-2-propyl[1,3]thiazolo[4,5-c]quinolin-7-yl)oxy]ethylcarbamate, 2-(1-methylethyl)thiazolo[4,5-c]quinolin-4-amine, 2-(2-methylpropyl)thiazolo[4,5-c]quinolin-4-amine, 8-methyl-2-propylthiazolo[4,5-c]quinolin-4-amine, 7-fluoro-2-propylthiazolo[4,5-c]quinolin-4-amine, 2-propylthiazolo[4,5-c[1,5]naphthyridin-4-amine, N-[3-(4-amino-2-propylthiazolo[4,5-c]quinolin-7-yl)phenyl]methanesulfonamide, tert-butyl 3-[(4-amino-2-propyl[1,3]thiazolo[4,5-c]quinolin-7-yl)oxy]propylcarbamate, N-{6-[(4-amino-2-propyl[1,3]thiazolo[4,5-c]quinolin-7-yl)oxy]hexyl}methanesulfonamide, tert-butyl 2-{2-[(4-amino-2-propyl[1,3]thiazolo[4,5-c]quinolin-7-yl)oxy]ethoxy}ethylcarbamate, or 7-[2-(2-chloroethoxy)ethoxy]-2-propyl[1,3]thiazolo[4,5-c]quinolin-4-amine.

13. The method of claim 1, wherein said agonist is ssRNA.

14. The method of claim 1, wherein said agonist is a compound or agent that binds to TLR8 thereby inducing signaling mediated by TLR8.

15. The method of claim 1, wherein said agonist is a compound or agent that induces the activity of a downstream signaling molecule that is activated by TLR8.

16. The method of claim 1, wherein the TLR8 agonist is a TLR8-selective IRM compound.

17. The method of claim 16, wherein the TLR8-selective IRM compound has a molecular weight of 1000 Daltons or less.

18. A method for preventing or treating an acute infection in a newborn comprising administering to said newborn an effective amount of a compound or agent that is an agonist of TLR8, wherein said agonist enhances the immune response of the newborn.

19. The method of claim 18, wherein said acute infection is a bacterial infection.

20. The method of claim 18, wherein said acute infection is a viral infection.

21. The method of claim 18, wherein said acute infection is a fungal infection.

22. The method of claim 18, wherein said acute infection is a parasitic infection

23. The method of claim 18, further comprising administration of an additional therapeutic agent.

24. A method for vaccinating a newborn against an infection or disorder comprising administering to said newborn an effective amount of a compound or agent that is an agonist of TLR7/8 and administering to said newborn a vaccine, wherein said agonist enhances the newborn's immune response to an antigen in said vaccine.

25. The method of claim 18 or 24, wherein said agonist is an imidazoquinoline compound.

26. The method of claim 18 or 24, wherein the imidazoquinoline compound is 4-amino-α,α-dimethyl-2-ethoxymethyl-1H-imidazo[4,5-c]quinolin-1-ethanol.

27. The method of claim 18 or 24, wherein said agonist is ssRNA.

28. The method of claim 18 or 24, wherein said agonist is a compound or agent that binds to TLR8 thereby inducing signaling mediated by TLR8.

29. The method of claim 18 or 24, wherein said agonist is a compound or agent that induces the activity of a downstream signaling molecule that is activated by TLR8.

30. The method of claim 18 or 24, wherein said agonist is administered concurrently with said vaccine or said therapeutic agent.

31. The method of claim 18 or 24, wherein said agonist is administered before said vaccine or said therapeutic agent.

32. The method of claim 18 or 24, wherein said agonist is administered after said vaccine or said therapeutic agent.

33. The method of claim 24, wherein said vaccine comprises a viral antigen.

34. The method of claim 24, wherein said vaccine comprises a bacterial antigen.

35. The method of claim 24, wherein said vaccine comprises a tumor antigen.

36. The method of claim 18 or 24, wherein the TLR8 agonist is a TLR8-selective IRM compound.

37. The method of claim 36, wherein the TLR8-selective IRM compound has a molecular weight of 1000 Daltons or less.

38. The method of claim 18 or 24, wherein the TLR8 agonist is a tetrahydroimidazoquinoline amine.

39. The method of claim 38, wherein the tetrahydroimidazoquinoline amine is 4-amino-2-(ethoxymethyl)-α,α-dimethyl-6,7,8,9-tetrahydro-1H-imidazo[4,5-c]quinoline-1-ethanol.

40. The method of claim 18 or 24, wherein the TLR8 agonist is a thiazoloquinoline amine.

41. The method of claim 40 wherein the thiazoloquinoline amine is 2-propylthiazolo[4,5-c]quinolin-4-amine, 2-propylthiazolo[4,5-c]quinoline-4,8-diamine, 2-butylthiazolo[4,5-c[1,5]naphthyridin-4-amine, N-{3-[(4-amino-2-propyl[1,3]thiazolo[4,5-c]quinolin-7-yl)oxy]propyl}-5-(dimethylamino)naphthalene-1-sulfonamide, tert-butyl 2-[(4-amino-2-propyl[1,3]thiazolo[4,5-c]quinolin-7-yl)oxy]ethylcarbamate, 2-(1-methylethyl)thiazolo[4,5-c]quinolin-4-amine, 2-(2-methylpropyl)thiazolo[4,5-c]quinolin-4-amine, 8-methyl-2-propylthiazolo[4,5-c]quinolin-4-amine, 7-fluoro-2-propylthiazolo[4,5-c]quinolin-4-amine, 2-propylthiazolo[4,5-c[1,5]naphthyridin-4-amine, N-[3-(4-amino-2-propylthiazolo[4,5-c]quinolin-7-yl)phenyl]methanesulfonamide, tert-butyl 3-[(4-amino-2-propyl[1,3]thiazolo[4,5-c]quinolin-7-yl)oxy]propylcarbamate, N-{6-[(4-amino-2-propyl[1,3]thiazolo[4,5-c]quinolin-7-yl)oxy]hexyl}methanesulfonamide, tert-butyl 2-{2-[(4-amino-2-propyl[1,3]thiazolo[4,5-c]quinolin-7-yl)oxy]ethoxy}ethylcarbamate, or 7-[2-(2-chloroethoxy)ethoxy]-2-propyl[1,3]thiazolo[4,5-c]quinolin-4-amine.


SEQUENCE LISTING (5 sequences)

SEQ ID NO: 1 - RNA, 20 nt, Artificial Sequence
  Description of Artificial Sequence Synthetic oligonucleotide
gcccgucuguugugugacuc

SEQ ID NO: 2 - DNA, 19 bp, Artificial Sequence
  Description of Artificial Sequence Synthetic primer
cctgagctccgattcatgt

SEQ ID NO: 3 - DNA, 22 bp, Artificial Sequence
  Description of Artificial Sequence Synthetic primer
ccctgatggtggctttcgtcaa

SEQ ID NO: 4 - DNA, 19 bp, Artificial Sequence
  Description of Artificial Sequence Synthetic primer
cgcatgacagcttctttga

SEQ ID NO: 5 - protein, 15 aa, Artificial Sequence
  Description of Artificial Sequence Synthetic peptide
SCGNNDESNISFKEK